(19) United States
(12) Patent Application Publication
(10) Pub. No.: US 2011/0230369 A1
(43) Pub. Date: Sep. 22, 2011

(54) METHOD FOR SCREENING COMPOUNDS FOR THEIR ABILITY TO INCREASE RIGIDITY OF RED BLOOD CELLS INFECTED BY A PROTOZOAN PARASITE OF THE GENUS PLASMODIUM, METHOD FOR FILTERING RED BLOOD CELLS, AND APPLICATION THEREOF

(76) Inventors: Pierre Antoine Buffet, Paris (FR); Guillaume Deplaine, Guyancourt (FR); Gencvièvc Milon, Paris (FR); Peter David, Paris (FR); Innocent Safeukui Noubissi, Paris (FR); Sylvie Perrot, Paris (FR); Valentine Brousse, Paris (FR); Narla Mohandas, New York, NY (US); Francois Lacoste, Neuilly Sur Seine (FR); Odile Puijalon, Issy Les Moulineaux (FR)

(21) Appl. No.: 12/994,600

(22) PCT Filed: May 28, 2009
(86) PCT No.: PCT/IB09/06194

§ 371 Date: Jun. 10, 2011

(30) Foreign Application Priority Data
May 28, 2008 (EP) 08157126.7

(51) Int. Cl. C40B 30/06; C12Q 1/02; C12N 5/071; C12Q 1/32

(52) U.S. Cl. 506/10; 435/29; 435/325; 435/26

(57) ABSTRACT

The invention relates to a method for screening compounds for their ability to increase rigidity of red blood cells (RBCs) infected by a protozoan parasite of the genus Plasmodium and in particular by Plasmodium falciparum. The present invention also relates to a method for filtering RBCs that enables retention, in a filtering unit, of RBCs having an abnormal and in particular a decreased deformability, as a surrogate for spleen filtering function. Said method enables in particular isolating and/or detecting Plasmodium-infected RBCs or spherocytes associated with acquired or hereditary spherocytosis from a sample of blood from a patient, or analysing in vitro spleen function of a patient. The invention also relates to the application of said methods, for the selection of compounds which selectively interact with iRBCs or selectively interact with ring-iRBCs and are suitable to increase their rigidity or to application of the method for filtering RBCs, for isolating and/or detecting RBCs with abnormal and especially reduced deformability. This new filtration method is also amenable to automation and allows the clearance or concentration of stiff RBCs, with wide experimental and medical applications in inherited or acquired RBC disorders (including malaria).

The present invention relates to a method for screening compounds for their ability to increase rigidity of red blood cells (RBCs) infected by a protozoan parasite of the genus Plasmodium and in particular by Plasmodium falciparum.

The present invention also relates to a method for filtering RBCs to that enables retention, in a filtering unit, of RBCs having an abnormal and in particular a decreased deformability. Said method enables in particular isolating and/or detecting Plasmodium-infected RBCs or abnormal RBC associated with acquired or hereditary spherocytosis, sepsis, hemoglobinopathies (alpha or beta thalassemia, sickle cell disease and sickle cell trait), auto immune hemolytic anaemia, other hemolytic anaemias, enzyme deficiencies (Glucose 6 Phosphate Deshydrogenase, Pyruvate Kinase, other red blood cell enzyme) and other red blood cell disorders with splenomegaly from a sample of blood from a patient, or analysing in vitro the spleen function of a patient.

The invention further relates to the application of said methods, for the selection of compounds which selectively interact with red blood cells infected by a protozoan parasite of the genus Plasmodium and are suitable to increase their rigidity.

Protozoan parasites of the genus Plasmodium cause diseases (malaria) in humans and in many animal species. In humans, malaria is mainly caused by Plasmodium falciparum, Plasmodium malariae, Plasmodium ovale, Plasmodium knowlesi and Plasmodium vivax. Plasmodium falciparum is the most common cause of disease and is responsible for about 80% of all malaria cases, and is also responsible for about 90% of the deaths from malaria in humans. Parasitic Plasmodium species also infect animals including birds, reptiles and mammals, in particular monkeys, chimpanzees and rodents. Human infections with several simian species of Plasmodium, namely Plasmodium knowlesi, Plasmodium inui, Plasmodium cynomolgi, Plasmodium simiovale, Plasmodium brazilianum, Plasmodium schwetzi and Plasmodium simium have also been documented.

Plasmodium falciparum initially infects the liver, but then moves into the blood, where it multiplies and persists through an asexual replication cycle in red blood cells (also known as RBCs, haematids or erythrocytes). After invasion of a red blood cell, Plasmodium falciparum undergoes continuous phenotypic change and several rounds of mitotic division. After approximately 48 hours, the infected red blood cell bursts and releases free parasites (merozoites), which either invade another red blood cell or are quickly (in less than half an hour) removed from the bloodstream by a variety of mechanisms.

The pathogenesis of malaria involves multiple parasite and host factors¹. Spleen filtering and immune functions have a major impact on the course of plasmodial infection in experimental models^{2,3}. In malaria-endemic countries, splenectomy predisposes to fever, to more frequent and higher parasitaemia (including circulating mature forms of the parasite), and may reactivate latent plasmodial infections^{4,5}. Despite relatively few published data (reviewed in⁵), clinicians include malaria in the list of infectious diseases justifying increased awareness in splenectomized non-immune patients⁶. Because key features may differ between animal and human plasmodial infection, and because detailed exploration of the human spleen is limited by ethical and technical constraints⁷, the fine interactions between Plasmodium falciparum-infected red blood cells (iRBCs) and the human spleen microcirculatory structures have been explored only indirectly^{8,9} or post-mortem¹⁰. Therefore, the mechanisms underlying the putative spleen protective or pathogenic effects during human malaria remain essentially speculative.

The human spleen senses moderate changes in RBC deformability, a mechanism leading to the selective physiological retention/destruction of senescent or abnormal RBCs^{11,48}. In several pathological conditions, RBC retention is associated with splenomegaly. Along the same line, splenectomy reduces anaemia in patients with various red blood cell disorders including hereditary spherocytosis, an inherited RBC disorder⁴⁹. Hereditary spherocytosis is characterized by an altered structural organisation of the RBC membrane—its cortical cytoskeleton included—, and splenic sequestration is the dominant mechanism responsible for reduced life span of RBCs (spherocytes). The severity of the disease in hereditary spherocytosis is directly related to the extent of decrease in membrane surface area and hence to a decrease in deformability⁵⁰.

Spleen-specific sensing of RBC deformability is operated by specific microcirculatory structures of its red pulp (RP). The best known deformability-sensing structure is the inter-endothelial slit (IES) in red pulp sinus walls. This is a dynamic 0.2-2 μm-wide (as estimated on optical and ultra-structural microscopic pictures from human spleen samples), 0.5-3 μm-long slit between 2 string-like endothelial cells, through which RBCs leaving the cords of the spleen red pulp must squeeze to reach the sinus lumen and the venous circulation^{11,21,51}. The IES is likely the most stringent RBC deformability-sensing structure in the body. To cross the narrow IES in walls of the venous sinuses, RBCs undergo considerable deformation: if cells are not sufficiently deformable, they are retained upstream from the venous sinus wall¹¹. Such RBC-processing functions are expected to act upon RBCs hosting Plasmodium¹².

The RBC is the main host cell for Plasmodium falciparum (asexual and sexual erythrocytic stage)⁵². Merozoites invade RBCs in which they develop for 48 hours, before giving rise to a new generation of merozoites. Parasite development inside the RBC results in an altered host-cell membrane, which presents new structural, functional and antigenic properties, some of which are related to the peculiar severity of Plasmodium falciparum infections^{13,14}. According to a widely accepted paradigm, RBC hosting asexual forms of P. falciparum (iRBC) circulate for 16-20 hours after invasion (ring stage), then sequester in the vasculature during the last 28-32 hours of the intra-erythrocytic cycle (trophozoite and schizont stage)³⁶. Hence, ring-iRBCs are the predominant Plasmodium falciparum forms found in the circulation, in contrast with tissue sequestration of cytoadhering mature-iRBCs. Deformability of mature-iRBCs is markedly reduced, which would result in the retention in the spleen of those escaping sequestration in “classical” vascular beds¹⁵. In addition, RBCs obtained from the blood of patients with malaria (P. falciparum-infected RBCs), then heat-stiffened ex-vivo, labelled and re-injected to the same patient were rapidly cleared by the spleen³⁹.

In this context, a quantified spleen circulatory framework is an essential prerequisite for a consistent understanding of malaria pathogenesis. Previous experimental determination of the proportion of spleen blood flow directed to the filtering beds of the open circulation in animals ranged from 90%¹⁶ to 10%¹¹, warranting direct in vivo explorations in humans.

We report here (and in⁵⁴) on complementary dimensions of human spleen physiology and malaria pathogenesis, following two conceptually connected approaches: in vivo imaging in healthy volunteers, and challenge of an ex vivo human spleen perfusion system with cultured iRBCs. We provide here the first in vivo confirmation of a two-compartment blood circulation in the human spleen, the slower compartment accounting for 10% of the flow. More than 50% of P. falciparum ring-infected-RBCs (rings) from in vitro culture is retained by isolated-perfused human spleens and accumulate along the abluminal side of sinus walls (i.e., upstream from inter-endothelial slits)⁵⁴. We show that, at each spleen passage, 10% of a previously ignored sub-population of ring-iRBCs is retained in the slow, open circulatory structures of the human spleen RP, most probably by a mechanical process. These results uncover heterogeneity of ring stage parasites and provide a new vision of RBC filtration by the spleen, shedding new light on control of parasite multiplication and RBC loss in malaria patients.

Unlike RBCs harbouring immature gametocytes—that are sessile—RBCs harbouring mature gametocytes (thereafter called mature gametocytes) circulate in the peripheral blood⁵³. Because mature gametocytes circulate, they become available to blood-feeding Anopheles sp. When mature gametocytes are contained in the blood meal of an Anopheles, this initiates the full development of the sexual stage of the parasite, a mandatory step toward transmission of P. falciparum to other human beings⁵².

Hence, presence of RBCs hosting rings or mature gametocytes in the peripheral blood of humans is an indication of a partial or absent spontaneous retention of the RBCs hosting these Plasmodium falciparum developmental stages in the spleen.

RBC deformability (and thus retention of RBCs in the spleen) depends (at least) on 3 factors: membrane viscoelasticity, cytoplasmic viscosity, and geometry of the cell, a marker of which is its surface area to volume ratio^{28,50}. RBC deformability can be estimated from the elongation index of a population of RBCs under shear stress with/without variation of osmolarity (ektacytometry, Lorca)⁵⁰. Time of transit through filters also reflects RBC deformability at a population level. These tools are therefore of limited use to study a sub-population accounting for less than 10% of the whole (this is most often the case for Pf-iRBCs in clinical or cultured samples). The micropipets and microfluidic devices disclosed in the prior art allow measuring the ability of RBCs to deform on a limited surface, or to flow through channels as narrow as 1 μm (but usually >5 μm-long)⁵⁵. Because their read-out is at a single-cell level, these tools are not well-suited for the fast analysis of large (>10³ cells) RBC populations. Because the peculiar deformability challenge upon RBCs and Pf-iRBCs crossing IES in vivo is not appropriately mimicked by available tools, no validated marker of RBC deformability that would predict spleen retention—including the stringent “inter-endothelial slit crossing challenge”—is available.

The present invention overcomes the above mentioned difficulties by the provision of a method for filtering RBCs that enables retention, in a filtering unit, of RBCs having an abnormal and in particular a decreased deformability. This method can be used for the detection of the presence of Plasmodium-infected RBC or of RBC of altered deformability (for example spherocytes) from a sample of blood from a patient, or for analysing in vitro spleen physiology in a patient.

In addition, stiff RBCs can be retrieved from the bead layers allowing further comparative analysis at the cellular, sub-cellular and molecular level to be performed with the different subpopulations available It is also amenable to automation and allows the clearance or concentration of stiff RBCs, with wide potential experimental and medical applications in inherited or acquired RBC disorders.

### BRIEF DESCRIPTION OF THE FIGURES

FIG. 1: Contrast ultrasound analysis of blood circulation in the human spleen parenchyma. (a) Enhancement of the ultrasound signal intensity in the spleen of a human volunteer receiving a constant perfusion of is Sonovue microbubbles. (b1) The ultrasound-induced decrease of signal intensity⁴³ in a sub-capsular zone [white line in (a)] was studied for ≧8 seconds. The best fit to the resulting Experimental signal-time Curve (EC) was obtained with a bi-exponential curve (TC), as shown on this typical example (the correlation coefficient R2 was >0.96 in all 16 volunteers). (b2) Graphic representation of the bi-exponential mathematical model of signal intensity versus time N(t)=V₁(C₀exp(−β₁t)+V₂(B+(C₀−B)exp(−β₂t), where the first and second terms correspond to a slow, and a rapid flow compartment, respectively. (c) Schematic representation of the blood circulation in the human spleen parenchyma derived from these observations.

FIG. 2: Clearance of iRBCs by the isolated-perfused human spleen. (a) Parasitaemia of Plasmodium falciparum FUP schizont-iRBCs (Δ), and ring-iRBCs () in the perfusate during 120 minutes of perfusion in 1 of 6 independent experiments with isolated-perfused human spleens (all 6 experiments shown in FIG. 7 a1-6). (b) Transmission electron microscopy of an end-experiment spleen sample showing a schizont-iRBC [S, b1-2, (bar=1 with knobs (b2, arrows) in the sinus lumen (SL, b1) of the red pulp, and a knobless ring iRBC (R, b1-3), in the cords (Co, b1). Another example of retained ring-iRBCs is shown in FIG. 4 (d1-2).

FIG. 3: Modeling of ring-iRBCs clearance during “circulation” and “circulation-recirculation” experiments. The uncovering of two ring-iRBC sub-populations. (a) Modeling of ring-iRBC parasitaemia in the perfusate, graphically illustrated by a theoretical example: one-compartment without a residual parasitaemia (a1), two compartments without a residual parasitaemia (a2, the plain line corresponds to the bi-exponential curve, the to dotted lines to the mono-exponential curves), and one-compartment with a residual parasitaemia (plateau phase, a3). The evaluation of the goodness of fit and the estimated parameters was based on the Akaike Information Criterion (AIC), the variability (VC) of the parameter estimates (the lower the AIC & VC values, the more parsimonious the model) and the random distribution of weighted residuals between measured and predicted concentrations with respect to time. Individual values are shown in Table 1. The AIC was lower for the two-compartment model and the 1-compartment model with residual parasitaemia than for the one-compartment model without a residual parasitaemia. However, for a two-compartment model the coefficient of variation of the second half-life parameter was not significant (>100%). So, the one-compartment model with residual parasitaemia was the best model. (b) “Circulation—recirculation” experiments. Part of the iRBC and RBC population prepared for a spleen challenge (“Spleen naïve” population) was kept at 37° C. in perfusion medium while the other part was perfused through the spleen. (b1). Forty minutes post the perfusion onset, iRBCs and RBCs were retrieved from the circulating perfusate (“Spleen passaged” population, b1). Both populations were differentially labeled using either PKH-26 or PKH-76, then pooled and reintroduced into the perfusate, 110-120 minutes post the initial perfusion step. Clearance kinetics of each iRBC subpopulation established using flow-cytometry showing that spleen “naïve” ring-iRBCs were cleared, whereas iRBCs previously submitted to 40 spleen passages were not. The most parsimonious model of the kinetics of the “spleen naive” population was again one-compartment with a residual parasitaemia. Mean (individual values) AIC and half-life from two independent experiments were similar to that observed during the 6 previous “Circulation” experiments (b2). The most parsimonious model for the “spleen passaged” population was a residual parasitaemia without elimination (see Table 1).

FIG. 4: Analysis of Plasmodium falciparum-IRBC deposition in the human spleen. (a1-2) Mean (SEM) number of iRBCs/100 RBCs in the peri-follicular zone (PFZ) or in the red pulp (RP) for ring-iRBCs (R, in PFZ, in RP), schizont-iRBCs (S, in PFZ, in RP), or Extra-Erythrocytic parasite Remnants (EER, in PFZ, in RP) subpopulations (mean from 6 isolated perfused human spleen experiments, Giemsa-stained sections, WP=White Pulp, bar=50_m). Typical aspects of each parasite development stage are shown on the inserts (middle column). (a3) Retention index [R1=(“number of iRBC/100RBC” as in a2)/(circulating parasitaemia at the end of the corresponding experiment)] for ring-iRBCs (R) or schizont-iRBCs (S) either in the PFZ or in the RP. A Retention Index of 1 (black interrupted line) corresponds to absence of retention. (b1-2) Same approach as in a1-2, but using PAS-staining (a=Central artery). (c1) Purple PAS-stained section (bar=5 μm) showing the basal membrane of red pulp venous sinuses. Working classification of RBC deposition in the spleen red pulp: in the sinus lumen (SL), in the cords in direct contact with the sinus wall basal membrane (cordal abluminal, CoA), or in the cords but without contact with the basal membrane (cordal stricto sensu, Co). (c2) Retention index for ring-iRBCs using the working classification of RBC deposition in the RP defined in c1. (d1-d2) Transmission electron microscopy of a human isolated-perfused spleen sample showing a ring-iRBCs (white star) and an uninfected RBC (black star) upstream and downstream of an inter-endothelial slit (black arrows), respectively. This disposition reflects the cord-(Co) to-sinus lumen (SL) circulation of RBC in the spleen red pulp. The knobless membrane of the ring-iRBC is very close to the sinus wall basal membrane (white arrows). For panels a, b & c: *, **, # mean statistically significant difference with p<0.05, p≦0.01 et #p<0.0001, respectively.

FIG. 5: Deformability of iRBCs. (a) Elongation index (EI) of red blood cells measured at different shear stresses (a1-2). (a3) EI of cultured ring-iRBC and schizont-iRBC at increasing parasitaemia at 30 Pascal (mean and standard deviation (SD) of 4 independent experiments). The mean [95% Confidence Interval (CI)] of the extrapolated EI of ring-iRBC at 100% parasitaemia is 0.47 [0.43-0.51]. (b) Scanning electron microscopy of spleen to samples processed at the end of the perfusion. Endoluminal view of sinus walls showing the typical string-like aspect of endothelial cells, with their nuclei (N) protruding in the sinus lumen. Cells are emerging from inter-endothelial slits (arrows, bar=3 μm). Upper left quadrangle: marked deformation of RBCs squeezing through inter-endothelial slits.

FIG. 6: Plasmodium falciparum-infected RBCs through the fast and slow circulatory compartments of the perfused human spleen. Schematic representation of observations in the ex-vivo human spleen model challenged with cultured iRBCs (see FIGS. 2-5), using the circulatory features extracted from in vivo imaging in human volunteers as a framework (FIG. 1). Ring- and schizont-iRBCs differed in their main phenotypic characteristics, ring-iRBC displaying no cytoadherent properties in vitro and no observable surface modifications. In the fast compartment, corresponding to the perifollicular zone on histological sections, only schizont-iRBC were retained, whereas both ring- and schizont-iRBcs were retained in the slow compartment, corresponding to the red pulp (b). This resulted in significantly different clearance rates. The similarity between the proportion of blood flowing to the slow compartment (10.2%) and the proportion of retainable ring-iRBCs retained in the same compartment (11%) was striking. The quantitative dimension of the spleen microcirculatory framework is important. Because, 5% of the cardiac output goes to the spleen, a given RBC will enter the spleen every 20 minutes. The slow compartment accounts for only 10% of the spleen plasma flow, which corresponds to 10-20% of spleen red blood cell flow (depending on the intensity of plasma skimming effect¹¹). Therefore, the quality control of the deformability of a given RBC occurs every 100-200 minutes. This fits with the 60-minute half-life of stiff heated RBCs previously observed in healthy controls³⁹. The order of magnitude of those previous clinical observations perfectly fits our framework.

FIG. 7: (a1-6) Parasitaemia of Plasmodium falciparum FUP schizont-iRBCs (Δtriangles), and ring-iRBCs ( circles) in the perfusate during 120 minutes of perfusion (6 independent experiments).

FIG. 8. (a) Surface immunofluorescence using a pool of hyperimmune sera from adult African patients (1/100 dilution) of cultured schizont-iRBC (a1) and ring-iRBC (a2) populations prepared for experiments, and counter-stained with Hoechst. The typical Giemsa-stained aspect is shown on inserts. Most schizont-iRBCs were intensely stained whereas no ring-iRBC was stained (bar=10 μm). (a3) PAGE auto-radiographs of Triton-SDS extracts from surface-iodinated cultured schizont-iRBCs (S), and ring-iRBCs (R). The 290 kDa band (arrow) probably corresponding to PfEMP1, was observed only in the schizont-iRBC extract.

FIG. 9. Schematic representation of experimental steps that can be carried out to screen for compounds increasing rigidity of Plasmodium falciparum ring-infected RBCs.

FIG. 10. Gravity-driven filtration with channel-perforated membranes: general set-up & experimental steps. 1. Filing the column with filtration medium (RPMI supplemented with 4% albumin and 5% Plasmion®, or PBS supplemented with 1% human albumin); 2. Blocking the filtering unit (with suspending medium supplemented with solubilized albumin) for 10 minutes prior to filtration; 3. Loading the column with sample (2%-2.5% hematocrit in filtering medium); 4. Filtration step; 5. Retrieval of samples upstream and downstream from the filtering unit; 6. Processing of samples for quantification of Pf-iRBC concentration and haemolysis.

FIG. 11. Channel-perforated membrane filtration. A-C, E. Aspect of the filtering unit that was used; A. Type of polycarbonate membrane used; B. Membrane cast; C. Membrane; E. Filtering unit connected to the column (ongoing filtration); D. Theoretical shape deformation of RBCs while crossing the Sterlitech° polycarbonate membrane; F. Supernatant colour of samples collected downstream from membranes with 2 and 3 μm-wide channels, respectively. The 2 μm sample shows mild to moderate haemolysis.

FIGS. 12 & 13. Bead-layer filtration. A. Bead source; B. Tips (Barrier tips 1000; Neptune) used to maintain bead layers; C. Loading a tip with beads of decreasing size before (C1) and after (C2) decantation; D. Schematic drawing of bead layers in a tip; E. Picture of a 7-mm 5-25 bead layer.

FIGS. 14 and 15. Shape deformation of RBC through natural and artificial inter-endothelial slits.

FIG. 14. A1. Cords (co) and sinus lumens (sl) in the splenic red pulp; A2-3. RBC squeezing (arrows) while crossing the sinus wall (Giemsa-stained slides from isolated-perfused human spleens). B&C. RBC squeezing (arrows) through inter-endothelial slits from cords to sinus lumen in isolated-perfused human spleens (transmission and scanning electron microscopy). D1-2. Schematic representation of RBC squeezing through polycarbonate membrane channels (D1) or inter-bead spaces (D2).

FIG. 15. A. Observed shapes of RBCs and endothelial cells (EC; dotted lines) at an inter-endothelial slit in the human spleen red pulp (transmission electronic microscopy); B. Geometric calculation of the greatest diameter of the narrowest strait in an inter-bead space (mixture of beads of equal size).

FIG. 16. Syringe-based hermetic circuit reference method for filtration—Principle.

FIG. 17. Syringe-based hermetic circuit reference method for filtration—Set-up for filtration in triplicate. 1. Electric syringe-pump with (from left to right) control screens for flow (in ml/minute), final volume (in ml) and pressure; 2. 3-way tubing; 3. Filters (arrows); 4. Collecting tubes.

FIG. 18. Centrifugation-based filtration. 1. Set-up of filtering unit in an Eppendorf tube; 2. Upstream sample in the filtering unit before centrifugation; 3. Filtering unit and Eppendorf tube in the centrifuge; 4. Aspect of filtering unit at the end of the first round of centrifugation; 5. Collecting tube after the first round of centrifugation (downstream sample).

FIG. 19. Liquid-phase fluorescence-based method for quantification of parasitaemia—Linear correlation between fluorescence intensity and parasitemia, as determined in parallel on Giemsa-stained smears or by liquid-phase fluorescence. Triplicate analysis of serial 2-fold dilutions of a cultured sample at 10% parasitemia.

FIG. 20. Liquid-phase fluorescence-based method for quantification of parasitaemia—Linear correlation between Giemsa-based and fluorescence-based estimates of the decrease (or increase) of parasitized RBC concentration. Upstream to downstream (lower part) or upstream to bead layer (upper part) analysis. X axis: retention rate in filter as assessed on Gielma-stained smears; Y axis retention rate in filter as assessed by liquid-phase fluorescence

FIG. 21. Sub-set from FIG. 20 Liquid-phase fluorescence-based method for quantification of parasitaemia—Detailed analysis of upstream to downstream retention rates, confirming the strong parasite stage dependence of retention in filters. X axis: retention rate in filter as assessed on Gielma-stained smears; Y axis: retention rate in filter as assessed by liquid-phase fluorescence.

### DETAILED DESCRIPTION OF THE INVENTION

The present invention relates to a method for screening compounds for their ability to increase rigidity of red blood cells (RBCs) infected by a protozoan parasite of the genus Plasmodium, said method comprising or consisting of the following steps:

a) culturing RBCs infected by said parasite and optionally and separately culturing uninfected RBCs, each culture being carried out both in the presence and in the absence of a compound to be tested for its ability to increase rigidity of iRBCs and;

b) measuring the deformability of one or several iRBCs cultured in the presence of said compound, and one or several iRBCs cultured in the absence of said compound; and,

c) optionally, measuring the deformability of one or several uninfected RBCs cultured in the presence of said compound, and one or several uninfected RBCs cultured in the absence of said compound, wherein a decrease by at least 5%, preferably at least 10% and more preferably at least 15%, in the deformability (in particular an increase by at least 5%, preferably at least 10% and more preferably at least 15%, in the rigidity) of iRBCs cultured in the presence of a compound in comparison with the deformability of iRBCs cultured in the absence of the same compound is indicative that said compound is able to increase rigidity of iRBCs.

In a particular embodiment, said method of screening comprises or consists of the following steps:

a) culturing RBCs infected by said parasite and separately culturing uninfected RBCs, each culture being carried out both in the presence and in the absence of a compound to be tested for its ability to increase and in particular selectively increase rigidity of iRBCs and;

b) measuring the deformability of:
one or several iRBCs cultured in the presence of said compound,
one or several iRBCs cultured in the absence of said compound,
one or several uninfected RBCs cultured in the presence of said compound, and
one or several uninfected RBCs cultured in the absence of said compound,
   wherein a decrease by at least 5%, preferably at least 10% and more preferably at least 15%, of the deformability of iRBCs cultured in the presence of a compound in comparison with the deformability of iRBCs cultured in the absence of the same compound is indicative that said compound is able to increase rigidity of iRBCs.

The “compound” as used herein can be any chemical compound, immunoglobulin (Ig), polypeptide, peptide, or other biotherapeutics that is able to increase rigidity of iRBCs. Chemical agents, referred to in the art as “small molecule” compounds are typically organic, non-peptide molecules, having a molecular weight up to 10,000, preferably up to 5,000, more preferably up to 1,000, and most preferably up to 500 Daltons. Ig can be generated using known methods and may be for example polyclonal, monoclonal, humanized or chimeric antibodies, single chain antibodies or Fab fragments. A “peptide” or “polypeptide” is any chain of two or more amino acids (2-20 amino acids for a “peptide” and more than 20 amino acids for a “polypeptide”), including naturally occurring or non-naturally occurring amino acid residues and amino acid residue analogues, regardless of post-translational modification (e.g., glycosylation or phosphorylation). Such a compound may be obtainable from or produced by any suitable source, whether natural or not. Where appropriate, said compound may be synthesized by any chemical or biological synthesis techniques.

According to a particular embodiment, a bank of compounds is screened. In particular, the bank from Sanofi Aventis can be screened.

According to a particular embodiment, the screened compounds are capable of interacting with the RBC membrane its cortical cytoskeleton included (the iRBC membrane its cortical cytoskeleton included at least) and/or entering into the RBCs (into the iRBCs at least), in particular crossing the RBCs lipid bilayer (the iRBC lipid bilayer at least), or interact with the modified bilayer itself.

According to a particular embodiment, the screened compounds selectively interact with iRBCs by increasing their rigidity. By “selectively interact with iRBCs” it is meant herein that the selected compounds interact with iRBCs and increase their rigidity whereas they do not increase the rigidity of uninfected RBCs and preferably of other types of cells. This means that one screens the deformability of uninfected RBCs cultured in the presence of the compound which is approximately the same or differs by less than 5% from that of the uninfected RBCs cultured in the absence of the compound.

In a particular embodiment, by “iRBCs”, or “Plasmodium-infected-iRBCs”, it is meant herein ring-RBCs (or ring-hosting RBCs) and/or gametocytes-hosting RBCs, in particular mature gametocytes-hosting RBCs.

According to a particular embodiment, the screened compounds selectively interact with ring-iRBCs and/or with gametocytes-hosting RBCs, in particular mature gametocytes-hosting RBCs by increasing their rigidity. By “selectively interact with ring-iRBCs and/or with gametocytes-hosting RBCs” it is meant herein that the selected compounds interact with ring-iRBCs and/or with gametocytes-hosting RBCs, and increase their rigidity whereas they do not increase the rigidity of uninfected RBCs nor preferably of other types of cells. This means that one screens (i) the deformability of schizont-iRBCs cultured in the presence of the compound which is approximately the same or differs by less than 5% from that of the schizont-iRBCs cultured in the absence of the compound, and (ii) the deformability of uninfected RBCs cultured in the presence of the compound which is approximately the same or differs by less than 5% from that of the uninfected RBCs cultured in the absence of the compound.

According to a particular embodiment, the screened compounds selectively interact with ring-iRBCs and/or with gametocytes-hosting RBCs, in particular mature gametocytes-hosting RBCs by increasing their rigidity. By “selectively interact with ring-iRBCs and/or with gametocytes-hosting RBCs” it is meant herein that the selected compounds interact with ring-iRBCs and/or with gametocytes-hosting RBCs, and increase their rigidity whereas they do not increase the rigidity of iRBCs at the schizont stage (schizont-iRBCs), nor the rigidity of uninfected RBCs nor preferably of other types of cells. This means that one screens (i) the deformability of schizont-iRBCs cultured in the presence of the compound which is approximately the same or differs by less than 5% from that of the schizont-iRBCs cultured in the absence of the compound, and to (ii) the deformability of uninfected RBCs or of other types of cells cultured in the presence of the compound which is approximately the same or differs by less than 5% from that of the uninfected RBCs or of other types of cells cultured in the absence of the compound.

By “ability to increase rigidity of iRBCs”, it is meant herein the ability to increase rigidity of iRBCs by at least 5%, preferably at least 10% and more preferably at least 15%. The ability of a compound to increase rigidity of iRBCs can be in particular assessed by measuring the deformability of iRBCs cultured in the presence and in the absence of said compound. Thus, according to a particular embodiment, “increase rigidity” means “decrease deformability” and in particular “decrease deformability by at least 5%, preferably at least 10% and more preferably at least 15%”.

By “infected by a protozoan parasite of the genus Plasmodium”, it is meant herein red blood cells that contain at least one parasite [i.e., a daughter cell of said protozoan parasite, which is the result of asexual reproduction), of the genus Plasmodium].

According to one embodiment, the parasite is chosen in the group consisting of Plasmodium falciparum, Plasmodium vivax, Plasmodium ovale, Plasmodium malariae, Plasmodium knowlesi, Plasmodium inui, Plasmodium cynomolgi, Plasmodium simiovale, Plasmodium brazilianum, Plasmodium schwetzi and Plasmodium simium, preferably in the group consisting of Plasmodium falciparum, Plasmodium vivax, Plasmodium ovate, Plasmodium knowlesi and Plasmodium malariae, and more preferably in the group consisting of Plasmodium falciparum and Plasmodium vivax.

In a particular embodiment, said parasite is Plasmodium falciparum, in particular the Palo Alto I strain of Plasmodium falciparum.

According to a particular embodiment, the parasite(s), in particular the merozoite(s) that have infected RBCs have developed into ring forms; hence, said infected RBCs are at the ring stage. In particular, fresh ring-infected RBCs (ring-iRBCs), i.e. ring-iRBCs of less than 14 hours, in RBCs of less than 8 days or of 8 days of age (preferably RBCs of less than 8 days of age), can be collected.

Alternatively or cumulatively, RBCs can be gametocytes-hosting RBCs, and preferably mature gametocytes-hosting RBCs.

Ring-iRBCs and/or gametocytes-hosting RBCs can be selected by artificially imposing synchrony upon developing Plasmodium parasites. Several methods are known in the art, for example the use of sorbitol or mannitol treatment; treatment of iRBCs with 5% sorbitol (for example 5 minutes at 37° C.) causes lysis of iRBCs containing late stages and preferentially selects for iRBCs with early ring stages. Treatment can be repeated (at least two times, for example, 2, 3, 4 or 5 times or more than 5 times), for example after 34 hours, to further select young stages and improve on the synchrony. Other techniques involve the separation of late-stage parasites, for example sedimentation in Plasmagel or gelatin. Thus, in one embodiment, step a) of the method of screening of the invention is preceded by a step of synchronization of the parasites of the iRBC culture.

According to one embodiment, RBCs (uninfected and/or infected RBCs) are human RBCs. RBCs from simian primates could also be used but human RBCs are more preferably used. Human RBCs of all blood groups are suitable for plasmodium growth, and are particularly suitable for Plasmodium falciparum growth. Type O RBCs are especially useful because of their compatibility with human serum or plasma of all other blood groups.

The screening performed can be a low throughput screening or a high throughput screening. Alternatively, the method of screening of the invention can comprises one or several step(s) of low throughput screening and one or several step(s) of high throughput screening.

RBC or iRBCs deformability can be defined as the ability to undergo substantial distortion without fragmentation or loss of integrity during microcirculation, and the ability to withstand the shear stress of the arterial circulation.

Hence, the measure of the deformability of RBCs and iRBCs is a phenotypical measure. It can be performed on a populational cellular scale and/or on an individual cellular scale. Thus, the method of screening of the invention comprises one or several step(s) of deformability analysis on a populational cellular scale and/or one or several step(s) of deformability analysis an individual cellular scale.

In order to enable assessment of the deformability of iRBCs, and in particular of ring-iRBCs and/or gametocytes-hosting RBCs, RBCs and iRBCs are generally cultured at 37° C. or 38° C., in RPMI 1640 medium or supplemented RPMI 1640 medium, such as complete medium (RPMI 1640 medium, bicarbonate, 25 mM glutamine, 0.2% glucose, 100 μM hypoxanthine, 10 μg/ml gentamicine and 10% AB+ inactivated human serum pool). In addition, these cells are preferably cultured in low oxygen pressure (low p0₂) conditions (generally 1-5% O₂), for example, in an atmosphere of 1% O₂, 3% CO₂ and 96% N₂.

RBCs and iRBCs can be incubated from 0.1 to 10 hours, preferably from 1 to 6 hours, and more preferably from 1 to 3 hours, for example 1, 2 or 3 hour(s) with the compound to be tested. Thus, in the method of screening of the invention, step b) can be performed from 0.1 to 10 hours, preferably from 1 to 6 hours, and more preferably from 1 to 3 hours, for example 1, 2 or 3 hour(s), after step a).

Deformability analysis is generally performed after (i) removal of the culture supernatant (for example by centrifugation at 1500 rounds per minute for minutes) and (ii) resuspension of the pellet in 1% albumin in PBS or 1% albumin in RPMI.

Various techniques in measuring the deformability of RBCs have been proposed, in particular ektacytometry, rheoscopy, microfluidics, micropipette aspiration and the use of optical tweezers. Thus, according to one embodiment, the deformability of RBCs and iRBCs is measured by means of a rheoscope, an ektacytometer, a microfluidic device (in particular a capillary microfluidic system), a micropipet and/or an optical tweezer.

Ektacytometry uses laser diffraction analysis of RBCs under varying shear stress levels. This technique has several advantages compared to other techniques, in that it is relatively easy to perform, has acceptable precision, can be performed at various shear stresses, and enables to measure rapidly cellular deformability using extremely small quantities of blood (less than 50 μl). Automated ektacytometers are commercially available. Models that can be used in the method of screening of the invention include LORCA (Laser-assisted Optical Rotational Cell Analyzer; Mechatronics, Hoorn, Netherlands) and RHEODYN-SSD (Myrenne, Roetgen, Germany). Ektacytometry provides a measure of cell deformability by determining the elongation index values of the cells under fluid shear stress using laser diffractometry and image analysis. RCBs are exposed to increasing shear stress and the laser diffraction pattern through the suspension is recorded. The laser diffraction pattern goes from circular to elliptical as shear stress value increases. From these measurements an elongation index for the cells can be derived.

According to a particular embodiment, the ektacytometer used to measure deformability of RBCs and iRBCs is the commercial rotating type ektacytometer, LORCA. Using such an ektacytometer, a viscous suspension of RBCs is sheared between two concentric cylinders. As indicated in the example part, the rotation of one of the cylinder causes deformation (elongation) of the RBCs. The laser beam diffraction pattern is detected with a video camera and analyzed by a computer which converts it into a digital value, the elongation index (EI).

Thus, in one embodiment, and in particular when an ectacytometer is used, the measure of deformability is carried out by reference to the elongation index (EI). This measure, which is generally defined as the ratio between the difference between the two axes of the ellipsoid diffraction pattern and the sum of these two axes, is usually determined from an intensity curve in the diffraction pattern using an ellipse-fitting program. Techniques for measuring RBC deformability often result in an indication of the mean value when deformability is analysed on a populational cellular scale.

In addition, in one embodiment and especially when an ektacytometer is used, deformability of RBCs and iRBCs, and in particular the elongation index, is usually measured over a range of shear stresses from 0 to 35 Pascal (Pa), preferably from 0.3 to 30 Pa, for example from 1.7 Pa to 30 Pa, and in particular at 1.7 Pa and/or at 30 Pa.

According to a particular embodiment, the parasitemia level of iRBCs is of at least 20% or 30% of the cell population, preferably at least 50% and more preferably at least 70% of the cell population.

By “parasitemia”, it is meant the quantitative content of parasites expressed in the percentage of RBCs containing at least one ring or gametocyte in the culture. The number of parasites can be counted, for instance, using an optical microscope, on a thin blood smear (for high parasitemias) or thick blood spot (for low parasitemias).

In general, and especially when deformability of RBCs and iRBCs is measured on a populational cellular scale (for example when an ektacytometer is used), the EI is measured at different parasitaemia (at least at two different parasitaemia), for example from 4% to 76% parasitaemia for rings-iRBCs, and extrapolated to 100% parasitaemia.

According to one embodiment, an EI below 0.43, more preferably below 0.42, for iRBCs cultured in the presence of the compound to be tested, when extrapolated at 100% parasitemia, at 30 Pa, is indicative that said compound is able to increase rigidity of iRBC.

Parasitemia can be induced or increased in a culture of RBCs (uninfected or already infected RBCs) by infecting (or re-infecting) said RBCs with a culture of iRBCs, in particular with a culture containing iRBCs at the to schizont stage. iRBCs at the schizont stage can be enriched in a culture of iRBC for example by suspension in a gelatinous solution (Plasmion® treatment) or by any other technique known in the art, in particular using a percoll gradient.

According to a particular embodiment, the iRBCs population used in step a) of the method of screening of the invention has been obtained by infecting uninfected RBCs, in particular fresh uninfected RBCs (i.e., RBCs of less than 8 days or of 8 days of age, preferably RBCs of less than 8 days of age) with a culture of iRBCs, and in particular with a culture in which the schizont-stage iRBCs have been enriched, for example by Plasmion® treatment. In a particular embodiment, deformability is thus measured using iRBCs which have only been submitted to culture and not to any other treatment.

Hence, according to a particular embodiment, the method of screening of the invention comprises or consists of the following steps:

1) enriching the schizont-stage iRBCs in a culture of iRBCs, for example by Plasmion® treatment or by any other technique known in the art;

2) infecting RBCs, in particular fresh RBCs (i.e., RBCs of less than 8 days or of 8 days of age, preferably RBCs of less than 8 days of age) with the schizont-enriched iRBCs culture of step 1) (this second step allows to obtain iRBCs at the ring stage);

3) culturing the iRBCs obtained at step 2) and, optionally and separately culturing uninfected RBCs, each culture being carried out both in the presence and in the absence of a compound to be tested for its ability to increase rigidity of iRBCs and;

4) measuring the deformability of one or several iRBCs cultured in the presence of said compound and one or several iRBCs cultured in the absence of said compound; and,

5) optionally, measuring the deformability of one or several uninfected RBCs cultured in the presence of said compound and one or several uninfected RBCs cultured in the absence of said compound,

wherein a decrease by at least 5%, preferably at least 10% and more preferably at least 15%, of the deformability of iRBCs cultured in the presence of a compound in comparison with the deformability of iRBCs cultured in the absence of the same compound is indicative that said compound is able to increase rigidity of iRBCs.

An example of such a method of screening is given in the example part (see example B).

In addition, in the above-mentioned particular embodiment, the parasites of the iRBC cultures, in particular the parasites of the schizont-enriched iRBCs culture can be synchronized by any method known in the art, in particular by any synchronization method cited herein, for example by one or several (2, 3, 4 or 5 or more than 5) sorbitol treatment(s). Said synchronization step or at least one synchronization step is preferably performed before the step of schizont-enrichment.

According to one embodiment, the method of screening of the invention further comprises a step of validating a screened compound with an isolated perfused human spleen model and/or an isolated perfused pig spleen model. Said further step is generally performed after the step(s) of deformability analysis.

According to a particular embodiment of the invention, in the method of screening disclosed above, the measure of deformability of RBCs or iRBCs is carried out using a method for filtering RBCs as defined herein, preferably by reference to the RBC or iRBC retention rate or retention ration (as described hereinafter).

The present invention also relates to the application of the method of screening of the invention, for the selection of compounds which selectively interact with iRBCs or selectively interact with ring-iRBCs and or with gametocytes-hosting RBCs (in particular with mature gametocytes-hosting RBCs) and are suitable to increase their rigidity.

The present invention also relates to a method for filtering RBCs that enables retention, in a filtering unit, of RBCs having an abnormal and in particular a decreased deformability, said method comprising or consisting of the following steps:
a) allowing a sample comprising RBCs to flow through a filtering unit; and
b) retrieving an aliquot of said sample before it flows through the filtering unit (upstream aliquot) and an aliquot of said sample after it has flown through the filtering unit (downstream aliquot); and
c) optionally retrieving the RBCs that have been retained into the filtering unit (retained aliquot); and
d) optionally analyzing the upstream and downstream aliquots and, optionally, the retained aliquot, and in particular determining the concentration or density of RBCs or of a RBC sub-population in the upstream and downstream aliquots, and, optionally, in the retained aliquot.

The method for filtering RBCs of the invention allows retention, in the filtering unit, of RBCs that would normally be retained in the spleen in vivo and thus reproduces the spleen filtering function that occurs in vivo, especially in humans.

By “decreased deformability”, (or increased rigidity or rigidification) it is meant herein a partial or total loss of deformability. The deformability is generally decreased by at least 5%, preferably at least 10% and more preferably at least 15%. In a particular embodiment of the invention, “decreased deformability” means “increased rigidity”.

The upstream, downstream, and retained aliquots are generally retrieved in microplates. Determination of the density can be performed in parallel for the upstream and downstream aliquots, by an automated method.

In a particular embodiment, step d) of the method for filtering RBCs to further comprises calculating the retention rate of RBC or of a RBC sub-population (i.e., percentage of RBCs or of a RBC sub-population retained) in the filtering unit, for example using the following formula:

(density of RBCs or of a RBC sub-population in the downstream aliquot−density of RBCs or of a RBC sub-population in the upstream aliquot)/density of RBCs or of a RBC sub-population in the upstream aliquot.

Alternatively, the method can comprise a step of comparing the density of RBCs or of a RBC sub-population in the downstream and upstream aliquots, for example by determining the retention ratio (density of RBCs or of a RBC sub-population in the downstream aliquot)/density of RBCs or of a RBC sub-population in the upstream aliquot).

In a particular embodiment, step d) comprises determining haemolysis in the upstream and downstream aliquots, and, optionally, in the retained aliquot, for example by quantifying human Lactate dehydrogenase (LDH) concentration in supernatant from centrifuged the upstream and downstream aliquots, and, optionally, from centrifuged retained aliquot.

In a particular embodiment of the invention, the RBCs are human RBCs.

Samples comprising RBCs that have an abnormal and in particular a decreased deformability are preferably used in the method for filtering RBCs of the invention. These RBCs include RBCs infected by a protozoan parasite of the genus Plasmodium, heated RBCs, and RBCs from patients who may have inherited or acquired immune dysfunctions as well as inherited or acquired RBC disorders, and preferably from patients who may have an hereditary or acquired disease, for example hereditary or acquired spherocytosis, elliptocytosis, sepsis, hemoglobinopathies (alpha or beta thalassemia, sickle cell disease and sickle cell trait), auto immune hemolytic anaemia, other hemolytic anaemias, enzyme deficiencies (Glucose 6 Phosphate Deshydrogenase, Pyruvate Kinase, other red blood cell enzyme)¹.

In a particular embodiment, the RBCs which are analyzed in step d) or at least one RBC sub-population which is analyzed in step d) are/is abnormal RBCs or Plasmodium-infected RBCs (iRBCs), and in particular ring-hosting iRBCs or gametocyte-hosting iRBCs, for example mature gametocyte-hosting iRBCs.

When iRBCs, and in particular ring-hosting iRBCs or gametocyte-hosting iRBCs are analyzed, step d) generally comprises or consists in:

determining the percentage of parasitaemia in the upstream and downstream aliquots, and, optionally, in the retained aliquot; and

optionally determining (i) the RBC retention rate in the filtering unit using the following formula: (percentage of parasitaemia in the downstream aliquot−percentage of parasitaemia in the upstream aliquot)/percentage of parasitaemia in the upstream aliquot or (ii) the retention ratio (percentage of parasitaemia in the downstream aliquot/percentage of parasitaemia in the upstream aliquot).

In a particular embodiment of the invention, the parasite is chosen in the group consisting of Plasmodium falciparum, Plasmodium vivax, Plasmodium ovale, Plasmodium malariae, Plasmodium knowlesi, Plasmodium inui, Plasmodium cynomolgi, Plasmodium simiovale, Plasmodium brazilianum, Plasmodium schwetzi and Plasmodium simium, preferably in the group consisting of Plasmodium falciparum, Plasmodium vivax, Plasmodium ovale, Plasmodium knowlesi, and Plasmodium malariae, and more preferably in the group consisting of Plasmodium falciparum (Pf) and Plasmodium vivax, for example a Plasmodium falciparum of the Palo Alto I strain.

In a particular embodiment of the invention, RBC of the sample have previously been exposed to a (known or novel) compound, in particular a compound that may be able to modulate and preferably to increase rigidity of RBCs, and for example a compound that may specifically modulate and preferably increase rigidity of iRBCs (in particular a compound specifically acting on ring-hosting iRBCs and/or on gametocyte-hosting iRBCs).

Small volume samples, for example samples of 1-100 microliters of packed red blood cells are generally used in the filtration method of the invention.

The sample is generally preferably suspended in a suspending medium comprising, consisting of or consisting essentially of PBS or RPMI supplemented with 4% albumin and 5% Plasmion® or with 1% albumax II®.

By “consisting essentially of”, it is meant herein that minor ingredients can be added in the sample without having a significant effect.

In a particular embodiment of the invention, the RBCs have been retrieved from a sample of blood (for example a sample of whole blood) and preferably from a sample of peripheral blood, previously obtained from a patient.

In another particular embodiment of the invention, the RBCs have not been retrieved from a sample of blood, but were preferably obtained by dilution of a sample of blood (for example a sample of whole blood) and preferably a sample of peripheral blood, previously obtained from a patient.

The RBCs used in the filtration method of the invention and in particular iRBCs can be collected from a sample of blood from a patient who has been treated with a compound that may have the ability to modulate and preferably to increase rigidity of RBCs as defined herein. For example, said RBCs can be collected after a few hours of therapy with said compound.

Alternatively, or cumulatively, RBCs used in the filtration method of the invention and in particular iRBCs can be obtained by in vitro culture.

In particular, drug-exposed RBCs can be collected after in vitro exposure to a compound that may have the ability to modulate and preferably to increase rigidity of RBCs as defined herein, in particular after in vitro culture of RBCs and in particular of iRBCs (for example ring-hosting iRBCs and/or gametocyte-hosting iRBCs) in the presence of said compound.

When a sample comprising iRBCs is used, a culture of iRBCs can prepared in vitro by infecting RBCs, preferably RBCs of less than 8 days of age, with iRBCs, in particular with a culture of iRBCs in which the schizont-stage iRBCs have been enriched, said enrichment being performed for example by Plasmion® treatment.

In a particular embodiment of the invention, the pores (or channels) of the filtering unit have a diameter in the range of 1 to 10 μm, and preferably in the range of 1.85 to 9.4 μm or 1 to 3 or 1 to 2 μm, for example a diameter of 2 μm.

In a particular embodiment of the invention, the channels of the filtering unit have a thickness of less than 24 μm, and preferably less than 5 μm.

In a particular embodiment of the invention, the flow through the filtering unit is driven by gravity, flush (for example by applying a constant pressure), aspiration or by centrifugation.

The filtering unit is usually placed in a column (for example when the flow through the filtering unit is driven by gravity or flush) or in a tube (for example when the flow through the filtering unit is driven by centrifugation).

In a particular embodiment of the invention, the hematocrit of the sample is low, for example less than 5%, and more preferably in the range of 2%-2.5%.

In a particular embodiment of the invention, the filtering unit comprises or consists of channel-perforated membrane(s), for example polycarbonate channel-perforated membrane(s). Channel-perforated membranes from Sterlitech Corporation in which channels diameter is in the range of 1 to 3 μm and channels length is 24 μm are particularly appropriate. For example, 2 μm-wide and 24 μm-thick polycarbonate channel-perforated membranes from Sterlitech Corporation can be used.

When channel-perforated membrane(s) are used, the flow through the filtering unit is generally gravity-driven. In particular, step a) can be gravity driven and performed under a constant pressure, for example a constant pressure of 80-85 cm of water, and preferably at a temperature of about 34-37° C.

Alternatively, the filtering unit can comprise or consist of one or several layer(s) of beads, and preferably of tin beads, wherein beads present in the filtering unit have a diameter in the range of 2-25 μm or 5-25 μm, and wherein channels (pores) formed by the inter-bead space within the filtering unit preferably varies between 0.74 and 9.4 μm or 1.85 μm and 9.4 μm.

In a particular embodiment of the invention, each layer of beads present in the filtering unit is at least 0.5-10 μm thick, the total thickness of beads in the filtering unit being of at least 5 mm, preferably 7 mm. For example, a layer of a thickness of at least 5 mm and preferably 7 mm, composed of a mixture of equal weight of beads the diameter of which is ranging from 5 to 15 μm and beads the diameter of which is ranging from 15 to 25 μm can be used. In a particular embodiment, a 7 mm-thick layer of beads the diameter of which is ranging from 5 to 25 μm is used. In another particular embodiment, the filtering unit comprises a 7 mm-thick layer of beads the diameter of which is ranging from 5 to 25 μm and a layer above comprising beads of lower diameter than 5 μm. In the filtering unit, the layers of beads are staked up on a filter suitable to maintain the beads and that is not involved in the retention capacity of the filtering unit.

When layer(s) of beads are used, the flow through the filtering unit is generally obtained using a syringe-pressured flow or by centrifugation (for example by centrifuging 1-2 minutes at 1500-2500 g). For example, an electric pump can be used to generate a constant 1 ml/min flow of suspending medium (for example PBS+1% Albumax II) through the layer during 8 minutes (i.e., a final volume of 8 ml). The upper pressure limit can be for example 999 mbars. Alternatively, the flow through the filtering unit can also be obtained using other techniques, and can, for example, be gravity-driven.

In a particular embodiment of the invention, layer(s) of beads are used and step a) is performed under a constant pressure, for example a constant pressure of 80-85 cm of water, and preferably at a temperature of about 20-25° C.

When the method for filtering RBCs of the invention comprises a step c) of retrieving the RBCs that have been retained into the bead layer(s), this step can be performed for example by differential decantation (e.g., after several steps of 1-3 minutes decantation by gravity).

Using the method for filtering RBCs of the invention, RBCs analysis in particular iRBCs analysis is performed on a populational cellular scale.

In a particular embodiment of the invention, step d) and in particular RBC density or parasitemia determinations, are performed by a liquid-phase fluorescence-based quantification method or after Giemsa staining. Fluorescence-based quantification of heated RBCs or parasitized RBCs can be performed by staining RBCs with PKH-26 or SYBR-I respectively. Fluorescence intensity is generally quantified using a counter, for example a FLX-800 counter.

Any of the methods disclosed herein can be performed in vitro.

In another aspect, the present invention relates to the application of a method for filtering RBCs as disclosed herein for screening compounds for their ability to modulate deformability and in particular to induce or increase rigidity of RBCs and especially of RBCs infected by a protozoan parasite of the genus Plasmodium, for example in a method for screening compounds for their ability to increase rigidity of Plasmodium-iRBC as disclosed herein. In a preferred embodiment, the compounds are screened on their capacity to increase the retention rate or the retention ratio of the infected RBC. The filtration method of the invention allows performing a low throughput screening or a middle or high throughput screening.

In a particular embodiment of this method of screening, the screened compounds are capable of interacting with the RBC membrane its cortical cytoskeleton included (the iRBC membrane its cortical cytoskeleton included at least) and/or entering into the RBCs (into the iRBCs at least), in particular crossing the RBCs lipid bilayer (the iRBC lipid bilayer at least), or interact with the modified bilayer itself.

In a particular embodiment of the invention, this method for screening compounds comprises or consists of the following steps:
applying the method for filtering red blood cells of the invention to two aliquots of a sample comprising RBCs, wherein only one aliquot has previously been exposed to a compound to be tested for its ability to modulate and preferably to increase rigidity of RBCs (the unexposed aliquot constituting an internal control); and
comparing the RBC retention rate or the retention ratio determined for both sample aliquots, wherein a variation of at least a 5%, preferably at least 10% and more preferably at least 15% between both RBC retention rates in absolute value or both retention ratios is indicative that said compound is able to modulate rigidity/deformability of RBCs and wherein an increase by at least 5%, preferably at least 10% and more preferably at least 15% in the RBC retention rate (in absolute value) or retention ratio obtained for the sample aliquot that has been exposed to the compound in comparison with the RBC retention rate or retention ratio obtained for the sample aliquot that has not been exposed to the compound is indicative that said compound is able to increase rigidity/decrease deformability of RBCs.

In a further aspect, the invention relates to the application of a method for filtering RBC as disclosed herein, for the selection of compounds which selectively interact with red blood cells infected by a protozoan parasite of the genus Plasmodium (iRBCs), and particularly interact with iRBCs at the ring stage and/or with gametocytes-hosting RBCs, in particular mature gametocytes-hosting RBCs, and are suitable to modulate and in particular to increase their rigidity (decrease their deformability) and also suitable to increase the retention rate or the retention ratio of these iRBC in the filtering unit according to the invention.

Hence, the filtration method of the invention can be used for screening compounds that are able to specifically decrease deformability (i.e., increase rigidity) of ring-hosting RBCs and/or of gametocytes-hosting RBCs (especially mature gametocytes-hosting RBCs) and thus induce, increase and/or quicken retention of these cells in the spleen and probably the spleen-dependent clearance of these cells from the circulating blood. The thus screened compounds are therefore useful for the treatment of malaria. Activity will be defined as the ability of a compound to induce or increase filtration-mediated iRBC retention.

In another aspect, the present invention relates to the application of the method for filtering RBCs as disclosed herein, for isolating and/or detecting RBCs with abnormal deformability and in particular with reduced deformability, for example for the in vitro diagnosis/detection of a clinical condition associated with an abnormal RBC deformability and in particular a decrease of RBC deformability.

In one particular embodiment of the invention, the method for filtering RBCs as disclosed herein, is used for isolating and/or detecting iRBCs and in particular ring-hosting RBCs and/or gametocytes-hosting RBCs (for example mature gametocytes-hosting RBCs) in a sample of blood from a patient that may have been infected by a protozoan parasite of the genus Plasmodium (for example Plasmodium falciparum).

In another particular embodiment of the invention, the method for filtering RBCs as disclosed herein, is used for isolating and/or detecting RBCs associated with an acquired or inherited disease, for the diagnosis or prognosis of acquired or inherited disorders that affect RBC deformability, for example hereditary or inherited spherocytosis (i.e., for isolating and/or detecting spherocytes), elliptocytosis, sepsis, hemoglobinopathies (alpha or beta thalassemia, sickle cell disease and sickle cell trait), auto immune hemolytic anaemia, other hemolytic anaemias, enzyme deficiencies (Glucose 6 Phosphate Deshydrogenase, Pyruvate Kinase, other red blood cell enzyme).

In another particular embodiment of the invention, the method for filtering RBCs as disclosed herein, is used for isolating RBCs with abnormal and especially reduced deformability for experimental studies.

The method for filtering RBCs of the invention can also be used for analyzing and/or isolating RBCs sub-populations, and in particular iRBCs, especially ring-hosting RBCs and/or of gametocytes-hosting RBCs.

In another particular embodiment of the invention, the method for filtering RBC as described herein is used for testing efficacity of anti-malarial drugs administered to a patient. The method of filtering is used with drug-exposed Plasmodium-infected RBC collected from the patient after a few hours or days drug administration and an observed variation in retention rate or retention ratio of Plasmodium-iRBC sampled before and after exposure to drugs indicates that drugs decrease deformability-dependent retention or destruction of Plasmodium-iRBC by spleen.

Finally, because experimental RBC filtration is the best available surrogate for spleen filtering function, it may provide the basis for a useful test for the in vitro analysis of spleen function in a patient, and in particular in a patient with inherited or acquired immune dysfunctions as well as inherited or acquired RBC disorders.

### EXAMPLES

### Example A

### Retention of Plasmodium falciparum Ring-Infected Erythrocytes in the Slow, Open Micro-Circulation of the Human Spleen

### Patients, Material and Methods

Contrast-enhanced ultrasonography in human volunteers. Sixteen 18-50 year-old healthy male volunteers were enrolled according to the following inclusion criteria: no risk factor for atheroma (i.e, normal arterial pressure, no present or past smoking habit, normal serum cholesterol level), no previous history of abdominal surgery, no present or past significant health problem (normal physical examination, normal serum creatinine, AST and bilirubin levels, and absence of antibody to HIV, HBV and HCV), normal splenic macrovascular structures as assessed by conventional ultrasonography and Doppler, and no clinical or biological indication of red blood cell disease—including normal blood cell count, serum haptoglobin, and hemoglobin electrophoresis. The study was approved by the Necker Hospital Investigational Review Board, and written informed consent was obtained from all participants. An ultrasound contrast agent (Sonovue°, Bracco, Milano, Italy) was injected intravenously at a constant infusion rate of 1 ml/min. Contrast-enhanced ultrasonography was performed using a Philips HDI 5000 (Philips U S, Bothell, Wash., USA). The spleen was imaged with a linear transducer (L10-5) using pulse-inversion imaging at low mechanical index to minimize microbubble destruction¹⁷, starting 2 minutes after the beginning of the infusion. Each acquisition was repeated 3 times and the raw data were transferred to a PC for further quantification. Each cineloop was quantified using HDI Lab (Philips U S, Bothell, Wash., USA), a software that takes into account the compression map and allows quantification in linear units. The signal intensity was calculated from a region-of-interest (ROI) located in the subcapsular area. The ROI position was moved to compensate for the respiratory movements and subcutaneous artefacts. The time-intensity curve was exported into Deltagraph (Red Rock software, Salt Lake City, Utah, USA). The data were fitted using a bi-exponential model. The quality of the fit was estimated using the correlation coefficient between the data and the model.

Human spleen retrieval. Spleens were retrieved and processed as described⁷. Medical and surgical care was not modified and patient written consent was obtained. The project was approved by the Ile-de-France II investigational review board. Upon a 30- to 90-minute period of warm ischemia linked to the surgical procedure, the main splenic artery was cannulated once the macroscopic aspect of the spleen had been examined by the pathologist, and a pre-experiment biopsy had been performed whenever required. The spleens were flushed with cold Krebs-albumin solution for transport to the laboratory.

Parasites. Plasmodium falciparum Palo-Alto (FUP-CB¹³), D10¹⁸, and FCR-3 were cultured as described¹⁹. Panning on human amelanotic melanoma cells (C32)²⁰ were repeated until a cytoadherent rate of more than 5 iRBC per C32 cell was obtained. Cytoadherent iRBCs were amplified, grossly synchronized by gel flotation until complete reinvasion occurred over less than 16 hours, then stored in liquid nitrogen as large >3 ml aliquots. Less than 7 days prior to the spleen challenge, aliquots were thawed, and parasites allowed to re-invade uninfected RBCs. Less than 14 hours prior to the spleen challenge, rings were eliminated by the gel flotation method, and mature forms allowed to re-invade in recently collected (<7 days) and washed RBCs so as to obtain 7-40 ml of packed RBCs at 2-7% parasitaemia, that were washed once in RPMI prior to introduction into the perfusate.

Ex-vivo spleen perfusion. Isolated-perfused spleen experiments were performed as described⁷, in a Plexiglas chamber maintained at 37° C. by a regulated warmed air flow. Briefly, once in the laboratory (cold ischemia time, 60-90 minutes), the spleen was connected to the perfusion device, and a progressive warming from 6-10° C. to 37° C. was performed by increasing the Krebs-albumin medium flow from 1 ml/min to 50-150 ml/min over 40 to 60 minutes. During this adaptation period the patient's RBCs were flushed from the spleen (haematocrit at the end of the warming period <0.1%). When the spleen temperature reached 35° C., uninfected O+ RBCs were added (final hematocrit level of 5-10%) and allowed to circulate for 30 to 60 minutes. Glucose, Na and K concentrations, O₂ and CO₂ partial pressures, and pH were monitored in the vein, artery, and reservoir every 10-30 minutes using a iStat device (Abbott laboratories, Abbott Ppark, ILL, USA). At steady state, key physiologic markers were maintained in the following ranges: perfusate flow 0.8-1.2 ml/gr. of perfused spleen parenchyma/min, temperature of the spleen capsule 36.7-37.2° C., Na 135-148 mEq/l, K 4-5 mEq/L, Glucose 4-12 mmol/l, pH 7.2-7.35, haematocrit 4-10%, and arterial-venous oxygen partial pressure decay 60-120 mm Hg. Finally, a mixture of >32-hour schizont-iRBCs (40±8 hrs post-invasion) and <14-hour ring-iRBCs (7±7 hrs post-invasion) at 0.2-6% parasitaemia was introduced in the circulating system for 2 hours after normal RBCs had been rinsed out for 5-10 minutes (haematocrit prior to the introduction of iRBCs <0.2%). Parasitaemia was quantified on Giemsa-stained thin smears or by flow cytometric analysis after staining of iRBC with Hoechst 33342 (diluted 1:1000; Molecular Probes, LSR, Becton-Dickinson, France). Data were analyzed using CELLQuest software (Becton-Dickinson, France).

Parasite staging and counting. The spleen tissue was fixed and processed exactly as described¹. The individual parasite nuclei, cytoplasm and malaria pigment were readily identified within the spleen tissue. iRBC stages were differentiated according to the following criteria. Ring-iRBC: light brown dot (nucleus)<1 μm with thin blue cytoplasm or pale round zone <½ erythrocyte size. Schizont-iRBC: light brown dot >1 μm or >1 brown dots with thick blue cytoplasm≧½ erythrocyte size. Extra-erythrocytic parasite remnant: brown dot(s) not surrounded by red staining. For each spleen slide, iRBCs were counted within both the perifollicular zone and the red pulp adjacent to 3 follicles. Follicles with a clear differentiation between the white pulp, the adjacent perifollicular zone and the red pulp were selected for counting. Ring- and schizont-iRBC were counted and localized on ˜150 photographs (at least 4000 RBCS) for each spleen.

Transmission and scanning electron microscopy. At the end of perfusion, spleen samples (1 mm³) were fixed overnight at 4° C. in 2.5% glutaraldehyde and 1% paraformaldehyde in 0.08 M cacodylate buffer supplemented with CaCl₂ (0.05%). For TEM, fixed samples were rinsed three times with 0.1 M sodium cacodylate buffer, postfixed with 1% osmium tetraoxide, 1.5% potassium ferricyanide in 0.1 M sodium cacodylate buffer for 1 hour at room temperature, and washed again with 0.1 M sodium cacodylate buffer. The samples were dehydrated through a graded series of ethanol bath (25% to 100%) and overnight in a mixture of Epon 812/propylene oxide at room temperature. After being embedded in Epon 812, samples were polymerized for 48 hours at 60° C. Ultrathin sections were prepared using a Leica ultracut UCT microtome and examined with a JEOL 1200 EX electron microscope operating at 80 kV. For SEM, fixed pieces were washed three times for 10 min in 0.1 M sodium cacodylate buffer, postfixed for 1 hour in 1% (w/v) osmium tetroxide, 1.5% potassium ferricyanide in 0.1 M sodium cacodylate buffer. Spleen sections were dehydrated through a graded series of 25%, 50%, 75% and 95% acetone solution for 10 minutes (each time). Samples were then dehydrated for 3×10 min in 100% acetone followed by critical point drying with CO₂. Dried specimens were sputtered with 22 nm gold palladium, examined and photographed with a JEOL JSM 6700F field emission scanning electron microscope operating at 5 kV or 7 kV. Images were acquired with the upper SE detector (SEI) and the lower secondary detector (LEI).

Measurement of RBC deformability. RBC and iRBC deformability were measured by ektacytometry using a laser-assisted optical rotational cell analyzer (LORCA®; Mechatronics, Hoorn, The Netherlands) as previously described²¹. The unit of RBC deformability, namely the elongation index (E.I.) was defined as the ratio between the difference between the two axes of the ellipsoid diffraction pattern and the sum of these two axes. Red blood cell deformability was assessed over a range of shear stresses (0.3 to 30 Pascal) including 1.7 Pa, which corresponds approximately to the intravenous stress on the arterial side of the circulation 14, and 30 Pa, which occurs in the sinusoids of the spleen where RBCs have to squeeze through the small intercellular gaps.

Erythrocyte surface immunofluorescence assay. This assay was performed in order to detect surface-iRBCs parasite proteins, as described. iRBCs in suspension in PBS were prepared from cultures. Labeling of RBC surface parasite antigen was performed with sera from hyperimmune African adults (a kind gift from P. Druilhe; 1:100 serum dilution in PBS/1% BSA) is followed by Alexafluor 488-conjugated goat anti-human affinity-purified IgG (diluted 1:200; Molecular Probes, Eugene, Oreg.). Parasite nuclei were stained with Hoechst 33342 (diluted 1:1000; Molecular Probes). Slides were mounted with Vectashield medium (Vector laboratories, Burlingame, Calif.). Images were acquired on a Zeiss Axiovert 200 M microscope, using an Axiocam HRc camera controlled by Zeiss Axiovision software (all from Carl Zeiss, Heidelberg, Germany).

Surface iodination of iRBCs. Highly synchronized mature stage iRBCs previously selected on cell C32 by panning procedure were enriched up to 80% by the gelatin flotation technique and then diluted with fresh erythrocytes (i.e., erythrocytes of less than 8 days or of 8 days of age, preferably erythrocytes of less than 8 days of age) to obtain approximately 20% ring-iRBCs at the next cycle. Surface iodination was done using the lactoperoxidase method²². The culture was stopped 14 h after reinvasion. Proteins were sequentially extracted with 1% Triton X-100, 2% SDS. Protease treatment of the samples (TPCK-treated trypsin and a-chymotrypsin TLCK (Sigma) was performed as described²². Iodinated samples were separated on a 5-17.5% gradient acrylamide gel. Autoradiography was done using Kodak Bio Max MS1 film. Pre-stained protein markers were purchased from Life Technologies (Gaithersburg, Md.) and New England BioLabs Inc. (Beverly, Mass.).

Cytoadherence assay. The cytoadherence of iRBC to C32 cells, which express both the putative receptor molecule CD36 and intercellular adhesion molecule 1 (ICAM-1) was studied as previously described²⁰. In brief, a monolayer of C32 cells was prepared in 25 cm3 cell culture flask (Corning Incorporated, USA). RBC suspended at a concentration of 5×106 iRBC/ml in cytoadhesion medium at pH 6.8, were added to cell culture flask and gazed (1% O₂, 3% CO₂, and 96% N₂) during 15 seconds, and this was incubated at 37° C. for 15 min with gentle rocking every 5 minutes. At the end of the incubation, the cell culture flask was gently rinsed four times in RPMI 1640 medium. The monolayer was fixed in methanol, stained with 2% Giemsa stain, and examined microscopically. The number of iRBC adherent to 1000 melanoma cells was counted. Results were expressed as the number of iRBC which adhered to 100 C32 cells.

Statistical analysis. We used the student's paired t-test for statistical analysis; p values <0.05 were considered significant. Compartment data analysis was performed using the WinNonLin software (version 5.1; Pharsight Corp., Mountain View, Calif., USA).

Results

Circulatory Compartments in the Human Spleen

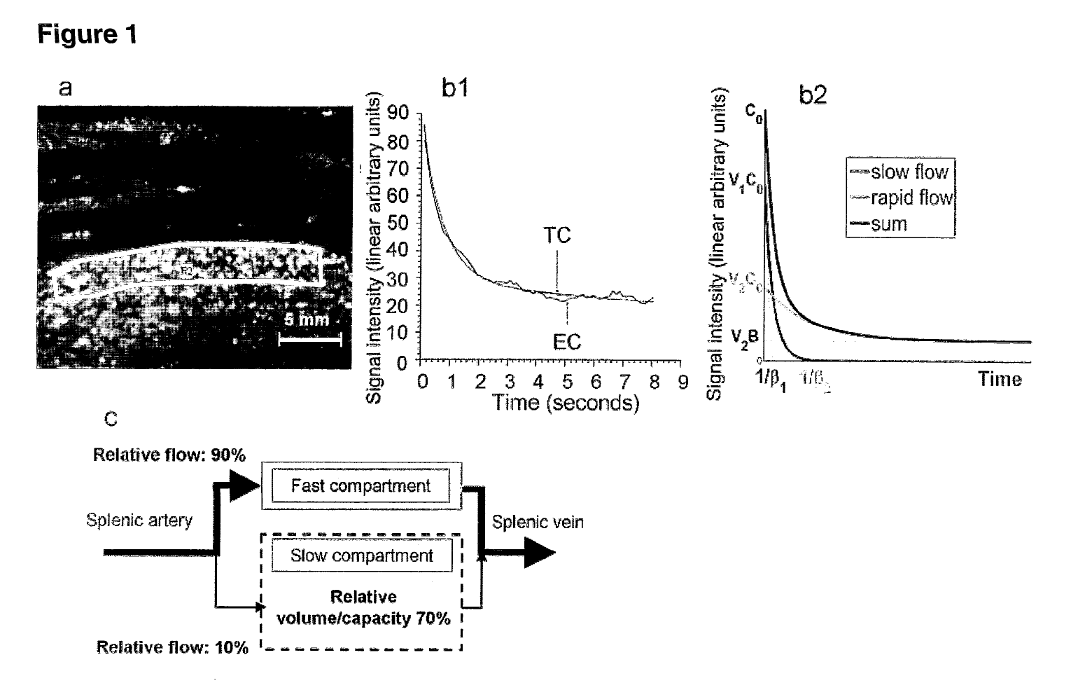

The circulatory pattern of the human spleen was explored using contrast-enhanced ultrasonography in 16 volunteers. Two minutes after starting the infusion of contrast agent (i.e, once a stable concentration of microbubbles was achieved), the spleen was studied using low mechanical index pulse inversion imaging. Despite the use of this low acoustic energy a certain amount of microbubbles was destroyed all along the continuous exposure of the spleen to the ultrasound beam. The time-intensity curve reflected the decrease in signal intensity (FIG. 1, Material & Methods section). The model that displayed the best fit with the experimental decay in the 16 volunteers combined two exponentials (correlation coefficient R2>0.96 in all cases). The bi-exponential function can be expressed as N(t)=V₁(C₀exp(−β₁t))+V₂(B+(C₀−B)exp(−β₂t)), where the first and second terms correspond to a slow, and a rapid flow compartment, respectively. C₀ refers to the initial (t=0) microbubble concentration, and was considered identical in both compartments. V₁ and V₂ refer to the relative fractional volumes (V₁+V₂=1), and (13₁ and 13₂ to the microbubble mean velocity in each compartment, respectively. Analysis of the experimental curves provided the following values: the Y intercept of each one-compartment exponential curve (Y1₀ and Y2₀) corresponds to C₀V₁ and C₀V₂, whereas 1/β₁ and 1/β₂ correspond to the exponential-characteristic times. In the slow compartment, the averaged (SD, range) relative fractional volume of the slow compartment was 70.6% (9.6, 51.5-84.4) and the averaged (SD, range) relative fractional flow was 10.12% (4.40, 3.99-16.47) (FIG. 1). Altogether, these observations strongly support the existence of a dual microcirculatory organization of the human spleen (FIG. 1), with approximately 10°/0 of the blood input flowing through the slow compartment (FIGS. 1&6).

Clearance of iRBC by Isolated-Perfused Human Spleens

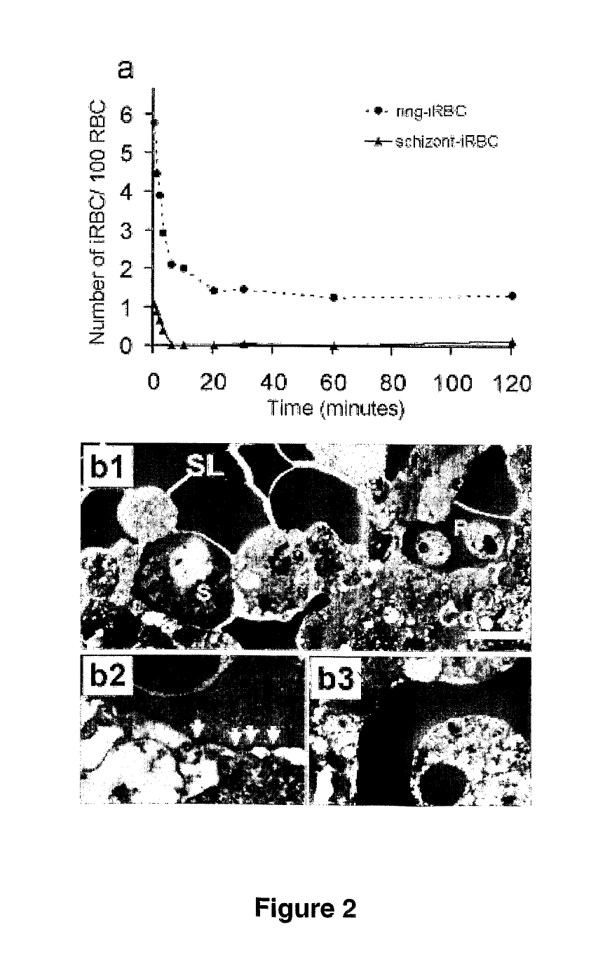

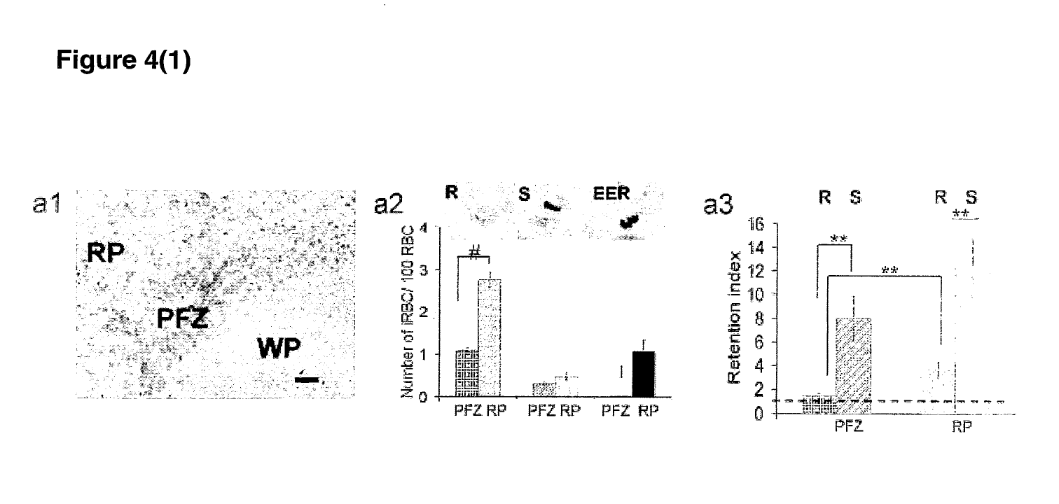

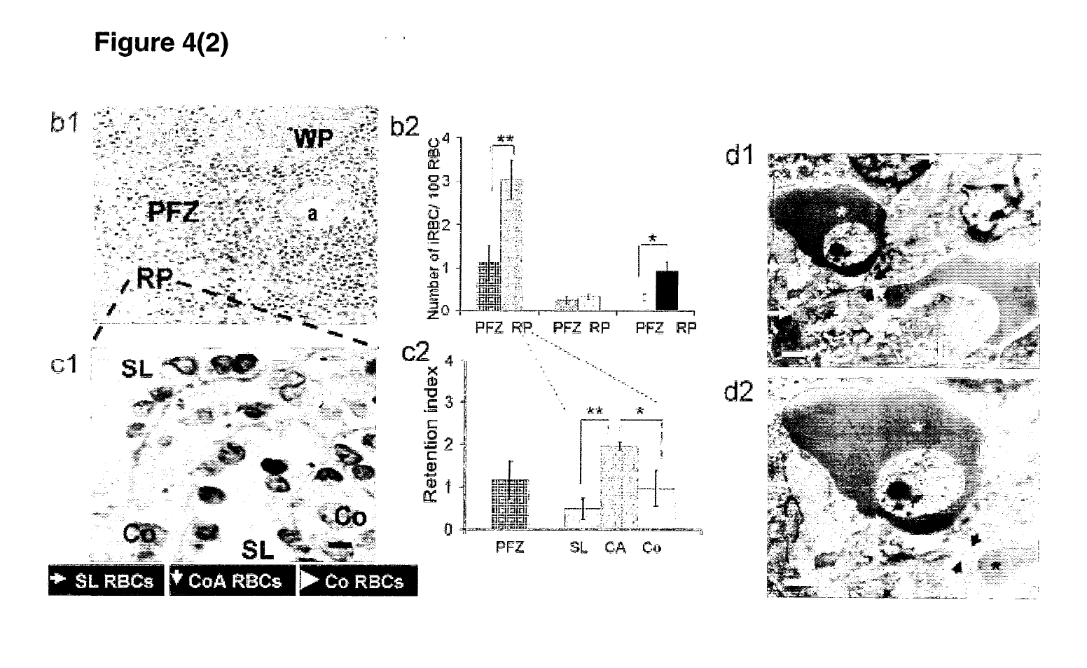

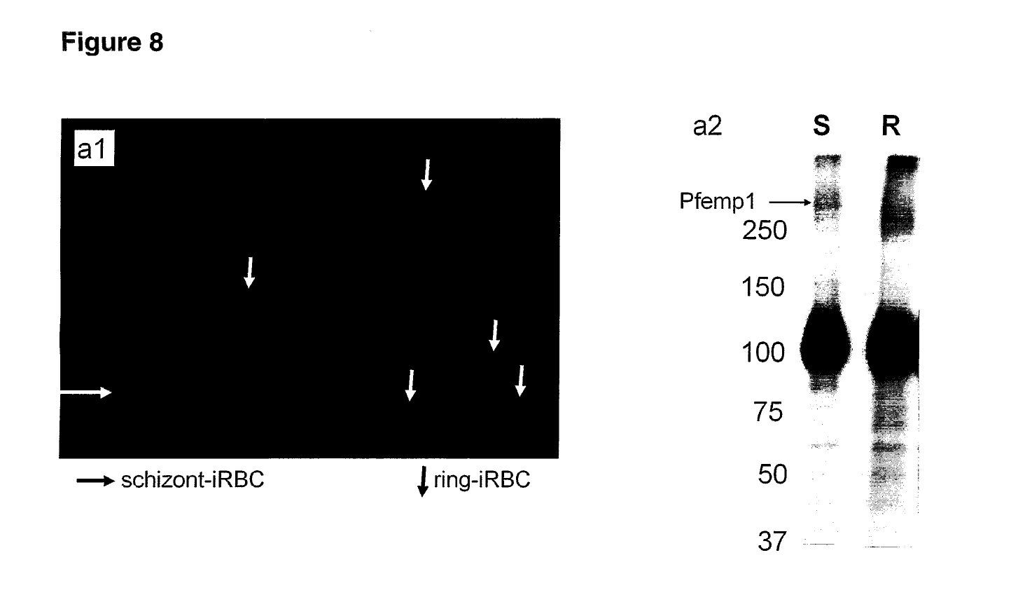

Isolated-perfused human spleens were perfused with highly synchronous parasite cultures at the schizont stage (40±8 hrs post invasion) or at the ring stage (7±7 hrs post invasion). Sequential Giemsa-stained thin films of the perfusate showed that circulating schizont-iRBC and—unexpectedly—ring-iRBCs parasitaemia rapidly decreased. Within 10 and 20 minutes, schizont- and ring-iRBC parasitaemias fell to 4.5% (range: 0-12.9) and 26.3% (range: 22.9-35.4%) of their initial values, respectively (FIG. 2a). Complete clearance of the schizont-iRBCs was rapid, while ring-iRBC parasitaemia decreased at a slower rate and reached a plateau (FIG. 2a). In contrast with schizont-iRBCs, ring-iRBCs displayed neither knobs nor iodinatable parasite proteins on their surface (FIG. 8), and no cytoadherence was noticed on C32 human melanoma cells (data not shown), nor on spleen histological examination (FIG. 4).

Heterogeneity of Ring-iRBCs Revealed by the Spleen Challenge

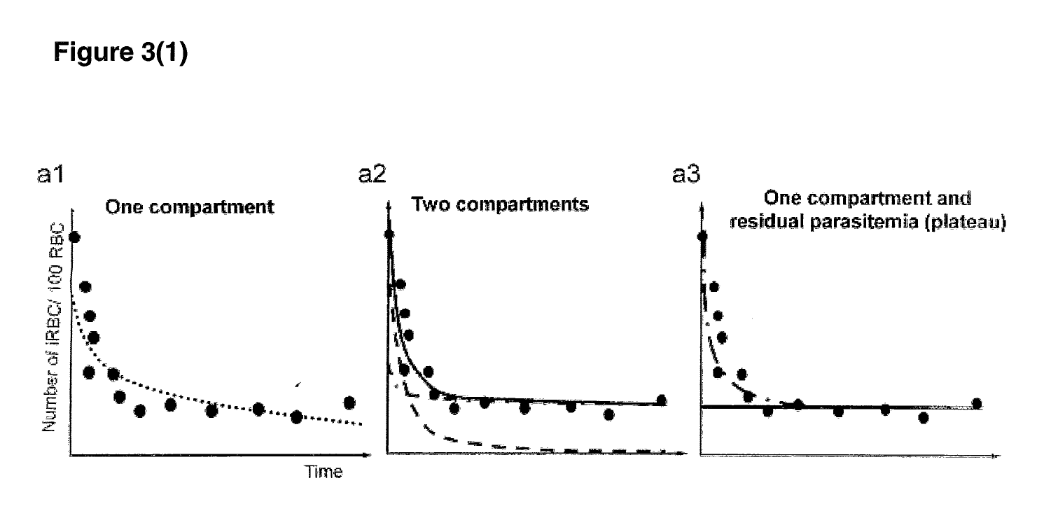

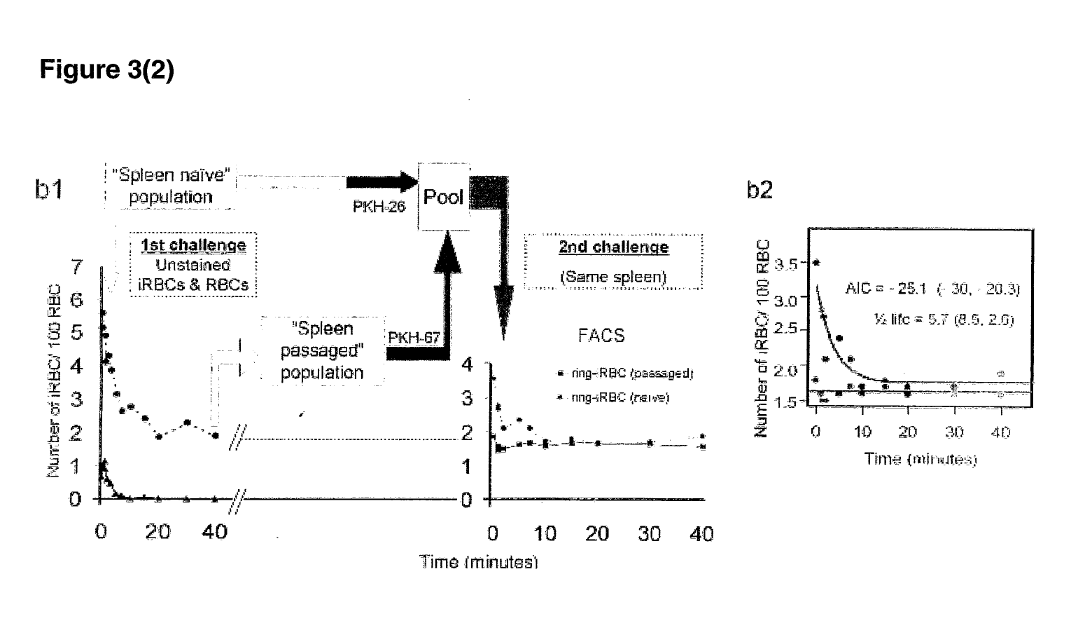

Partial clearance of ring-iRBCs could reflect their slower circulation through the spleen or their stable retention in the spleen. To clarify this issue, ring-iRBCs preparations were split, with one part immediately perfused in the spleen and a second part kept at 37° C. in Krebs-albumin medium (control “spleen naïve” cells). Forty minutes (i.e. 40 spleen passages) after starting perfusion, the unretained cells were collected from the perfusate. These “spleen passaged” and the “spleen naïve” populations were labeled with a different PKH each, pooled and reintroduced into the spleen. Sequential samples from the perfusion system were next analyzed using flow cytometry (FIG. 3). The mean (SD) half-life of “spleen-naïve” ring-iRBCs (5.7±3.1 minutes, two independent experiments) was similar to that observed during 6 previous experiments (see next section of the results “Modelling iRBC clearance”). In contrast, the “spleen passaged” ring-iRBCs were not cleared (FIG. 3 b, c). This indicates that ring-iRBCs indeed consist of two distinct subpopulations, one retained in the spleen and one flowing through. This heterogeneity of the ring-iRBCs population with regard to spleen retention was stable over time in our culture conditions, and fairly independent of the initial parasitaemia introduced in the perfused spleen (FIG. 2a). It was also independent of the parasite strain, since similar results were obtained with D10.

| | | | Estimated parameters | | | |
|---|---|---|---|---|---|---|
| | | | Phase 1 | | Phase 2 | |
| Experiment | Model | AIC | Half-life (mn) | CV (%) | Half-life (mn) | CV (%) |
| a | One-compartmental | 20.7 | 64.4 | 23 | | |
| | One-compartmental + plateau | −7.3 | 5.4 | 17 | | |
| | Two-compartmental | −5.0 | 5.5 | 24 | 8216.6 | 3166.6 |
| b | One-compartmental | 9.1 | 70.5 | 18 | | |
| | One-compartmental + plateau | −19.0 | 2.8 | 17 | | |
| | Two-compartmental | −19.4 | 2.4 | 19 | 370.1 | 70 |
| c | One-compartmental | 6.0 | 62.5 | 31 | | |
| | One-compartmental + plateau | −9.6 | 4.8 | 25 | | |
| | Two-compartmental | −7.5 | 4.9 | 34 | 2571.8 | 1135 |
| d | One-compartmental | 0.8 | 59.7 | 20 | | |
| | One-compartmental + plateau | −12.9 | 5.2 | 26 | | |
| | Two-compartmental | −19.1 | 2.9 | 28 | 167.2 | 33 |
| e | One-compartmental | −8.9 | 90.0 | 19 | | |
| | One-compartmental + plateau | −17.7 | 14.5 | 29 | | |
| | Two-compartmental | −15.8 | 14.2 | 66 | 1065.2 | 756 |
| f | One-compartmental | 6.0 | 121.9 | 38 | | |
| | One-compartmental + plateau | −23.5 | 2.8 | 23 | | |
| | Two-compartmental | −21.4 | 2.7 | 28 | 28286 | 5603 |
| e′ | One-compartmental | −25.5 (−13.5) | 66.9 (103.5) | 19 (55) | | |
| | One-compartmental + plateau | −30.0 (−11.7) | 8.8 (14.5) | 42 (199) | | |
| | Two-compartmental | −28.5 (−12.0) | 8.5 | 156 | 204400 | 507876 |
| f′ | One-compartmental | −7.1 (−34.3) | 50.5 (8968.9) | 35 (1819) | | |
| | One-compartmental + plateau | −20.3 (−32.3) | 2.6 (37687) | 41 (>1010) | | |
| | Two-compartmental | −18.3 (−36.3) | 8.5 | 63 | 2534 | 2342 |

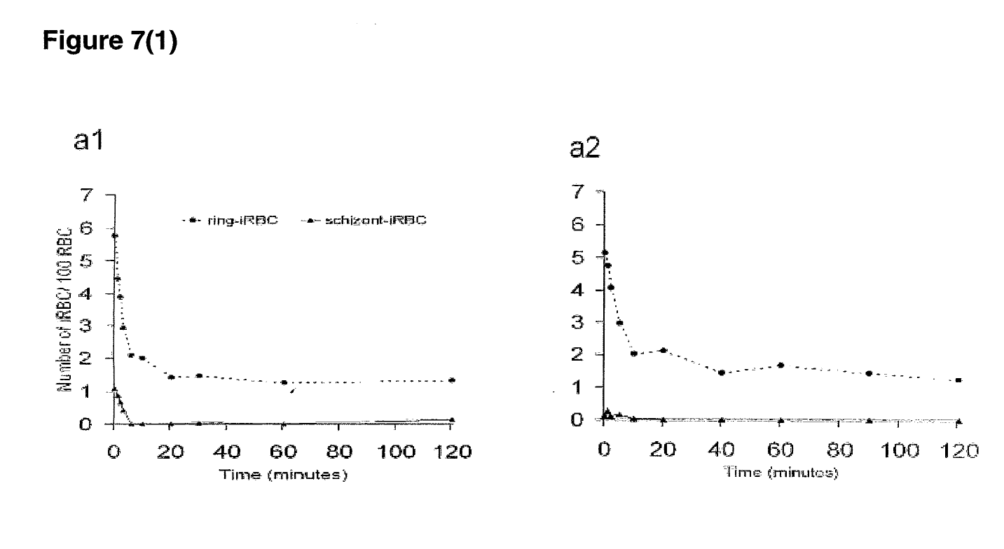

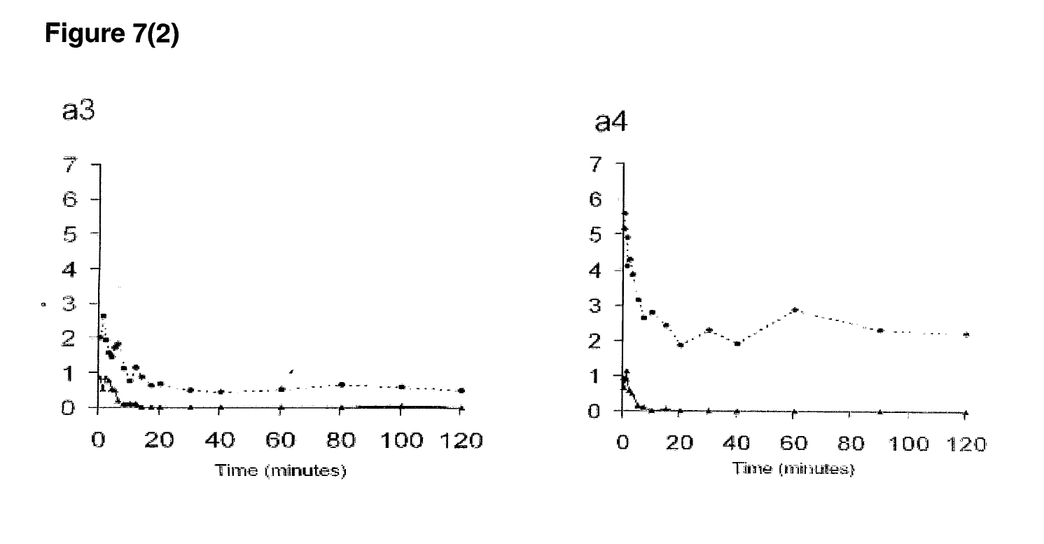

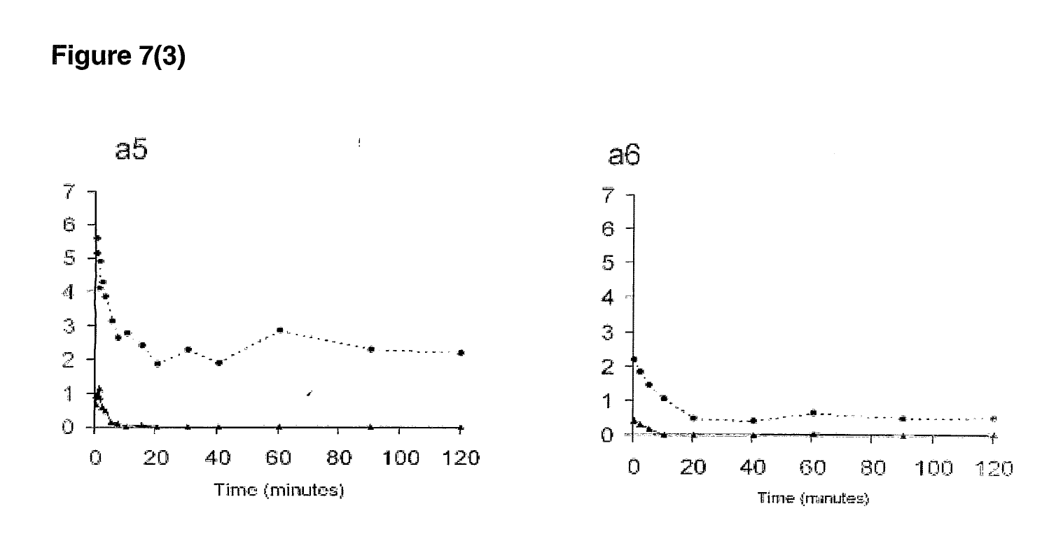

FIG. 3a1-3. Data are from circulation experiments (a-f, FIG. 2 and FIG. 7) or circulation-recirculation experiments (e′ and f′, FIG. 3b1 & b2).

Modelling iRBC Clearance

The kinetics of iRBC parasitaemia in the perfusate was modeled using either a one- or two-compartment model, without or with a residual parasitaemia (plateau-phase) (FIG. 3a1-3). A one-compartment model without a residual parasitaemia had the best performance to describe schizont-iRBC kinetics (FIG. 3a3). The kinetics of ring-iRBCs in the perfusate was best described by a one-order clearance phase of a “Retainable” subpopulation (˜74% of input) alongside a free circulation of a “Flow-Trough” subpopulation (˜26% of input) (FIG. 3 and Table I).

This model fitted with circulation-recirculation observations (see previous section) and moreover allowed a simple determination of the proportion of retained iRBCs. This is consistent with initial heterogeneity of the population of ring-iRBCs used for challenge evidenced above. The mean clearance half-life of the “Retainable” ring-iRBCs subpopulation (5.9 minutes, CI95°/0:2.0-9.4), was ˜2-fold higher than the clearance half-life of schizont-iRBCs (2.8 minutes, CI95%; 1.5-3.9), suggesting at least partially different respective clearance mechanisms. The 5.9 minute clearance half-life of the retainable ring-iRBCs fits with the removal of ˜11% of the retainable input at each spleen passage, assuming that each RBC crossed the isolated-perfused is human spleen every minute⁷.

Retention of Ring-iRBCs in the Human Spleen

At the end of the experiments, the spleen tissue was processed for histological analysis and the distribution of ring- and schizont-iRBCs in the RP and perifollicular zone (PFZ) was quantified (FIG. 4). The ring-iRBC tissue parasitaemia was significantly higher in the RP than in the PFZ both in Giemsa-stained (2.8% v. 1.1%, p=0.00009, FIG. 4a1-2) and Periodic Acid Schiff (PAS)-stained (3.0% vs.1.1°/o, p=0.007, FIG. 4b 1-2) sections. The mean (CI₉₅%) ring-iRBC retention index defined as the ratio (“tissue parasitaemia”)/(circulating parasitaemia at the end of the experiment) was significantly higher in the RP (3.7; 1.9-5.4), than in the PFZ (1.4; 0.8-2.0, p=0.002, FIG. 4a3). The mean overall retention index of ring-iRBCs in the spleen (including both zones) was 2.5. Interestingly, the retention index of ring-iRBCs in the PFZ was close to 1, suggesting that—as previously observed with artesunate-exposed iRBCs⁷—the PFZ is more a transit zone than a retention/processing area for ring-iRBCs. The mean retention index of schizont-iRBCs was high (>8) in both zones with a trend toward stronger retention in the RP. Use of PAS, which intensely stained the peculiar helix-shaped basal membrane of venous sinuses²³, allowed analysis of the distribution of ring-iRBCs in the sub-compartments of the RP, specifically, the cords, the sinus lumens and the abluminal side of the sinus walls (FIG. 4c1). Most (86.0±6.9%) ring-iRBCs were observed in the cords, whereas 14.0±6.9% were in the sinus lumens. Significantly more ring-iRBCs were in direct contact with the PAS-stained basal to membrane of venous sinuses (ie, immediately upstream of the narrow inter-endothelial slits), than in other sub-compartments of the RP (FIG. 4c2). The very close proximity of knobless ring-iRBCs to the sinus basal membrane and inter-endothelial slits was confirmed by transmission electron microscopy (FIG. 2b3 and FIG. 4d1-2).

Deformability of iRBCs

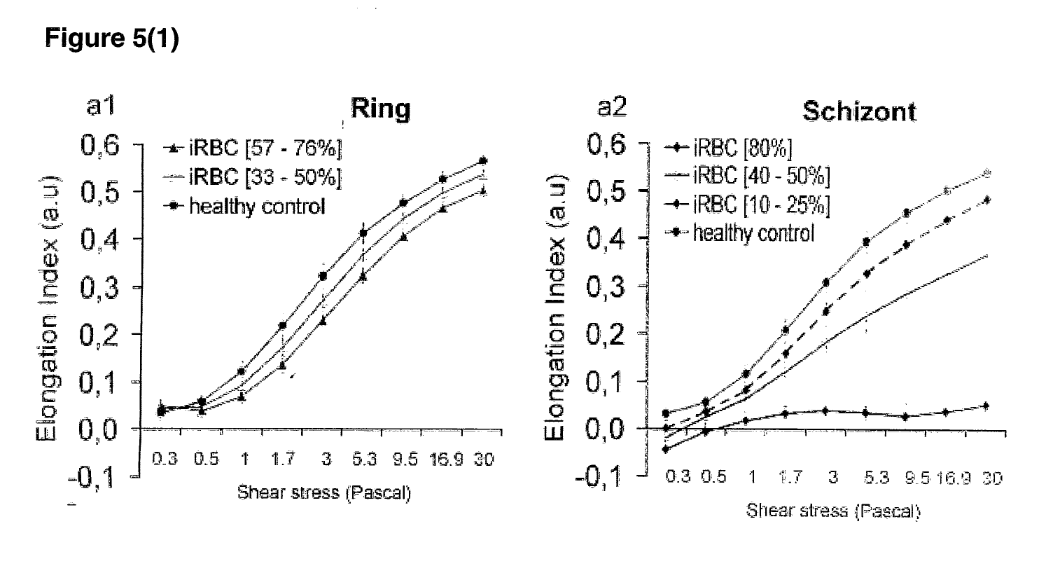

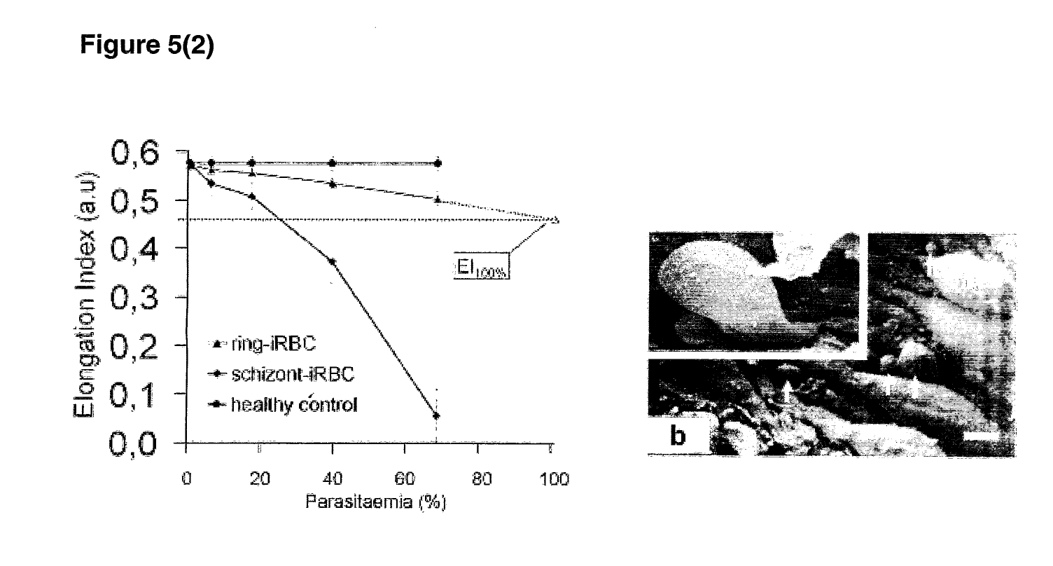

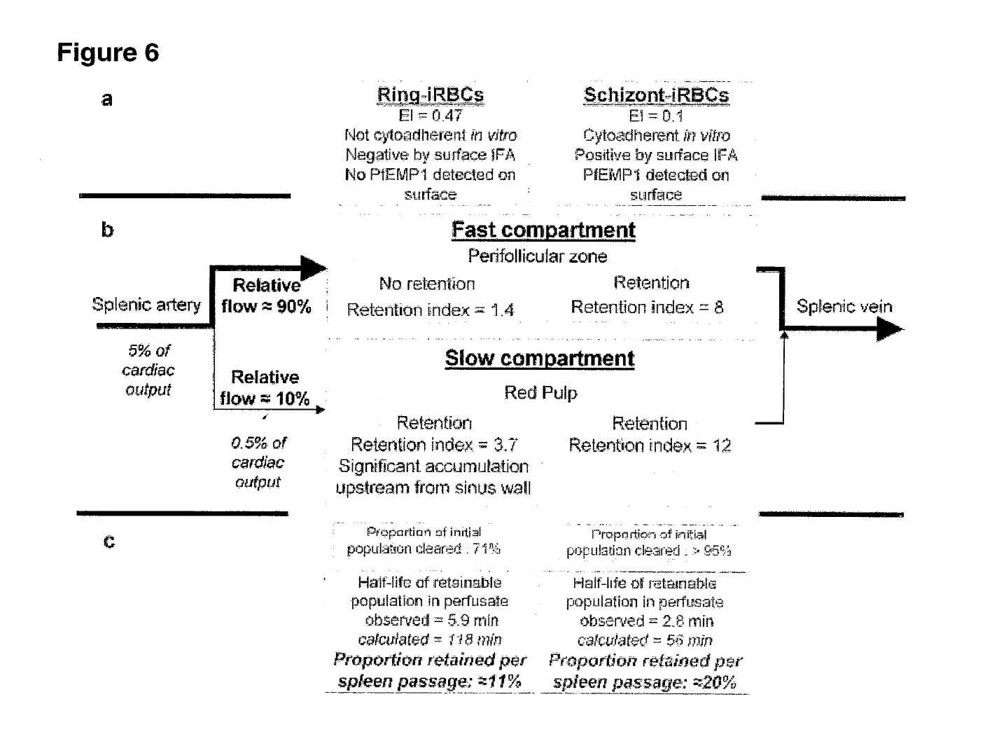

The elongation index (EI) of ring- or schizont-iRBCs populations was measured using LORCA. In order to infer the deformability at the level of a single iRBC, we measured the EI of the synchronized populations at different parasitaemia (3% to 80% for schizonts, 4% to 76% for rings, FIG. 5A) and extrapolated to 100% parasitaemia. As expected, schizont-iRBCs had a very low elongation index (<0.1 at 30 Pascal, 80% parasitaemia) across all shear stresses. At 30 Pascal (similar to that encountered in the splenic sinusoids²⁴), ring-iRBCs were moderately but significantly less deformable than co-cultured co-perfused uninfected RBCs (FIG. 4 A). When extrapolated to 100% iRBC population, the mean (CI₉₅%) EI of ring-iRBC was 0.47 (0.43-0.51), vs 0.58 for control uninfected RBCs (p<0.0001 FIG. 4 A3).

Discussion

The safe approaches used here provide novel, interconnected insights into the human spleen physiology and malaria pathophysiology, and raise a new paradigm whereby the ring stage population is heterogeneous, the less deformable subset being retained in the spleen. Contrast-enhanced ultrasonography showed a two-compartment microcirculatory organization, with 10% of the blood input flowing through the slow compartment. A substantial proportion of ring-iRBCs was rapidly cleared by isolated-perfused human spleens. The 5.9 minute clearance half-life of the retainable ring-iRBC population fits with the removal of −10% of the input at each spleen passage, consistent with their retention in the slow circulatory compartment. In keeping with that interpretation, ring-iRBCs accumulated only in the RP, along the abluminal side of sinus walls. The deformability of ring-iRBCs was moderately but significantly reduced. Those rings were tiny, lacked knobs, did not cytoadhere in vitro and did not display any observable parasite-induced surface modification, excluding a PfEMP1-mediated retention mechanism. The overall picture indicates retention of ring-RBCS with reduced deformability upstream from the narrow inter-endothelial slits (i.e, the microcirculatory structures of the slow compartment that stringently challenge RBC deformability^{11,25}) likely reflecting an original mechanism of micro-organism clearance, based on the new biophysical properties of the host cell.

A few exceptions apart^{20,26}, ring-iRBCs have been considered an exclusively circulating component of the total body parasite biomass during human Plasmodium falciparum infection²⁷. Their reduced deformability has been known for decades^{15,28}, but deemed too mild to trigger spleen retention. However, experimental and clinical observations support the hypothesis that this phenomenon does occur during the course of acute human malaria. The ex-vivo spleen model filtered drug-exposed iRBCs at a rate similar to clearance rate in patients⁷, and the efficient clearance of schizont-iRBCs observed here further validates this experimental set up. Importantly, a recent study of post-mortem samples from patients who died from P. falciparum-induced severe malaria reported unexplained spleen retention of knobless iRBCs, with a mean sequestration index of 2.19 in the spleen (compared to 0.63 in the liver)²⁹, a figure in line with the retention index of 2.5 determined here. We thus interpret these post-mortem observations as evidence of young ring-iRBC spleen retention in these patients. Another puzzling aspect of malaria pathogenesis can be revisited in light of our findings. The parasite biomass in Plasmodium falciparum-infected patients, as calculated from circulating ring-iRBCs was 2-fold lower than calculated from plasma levels of HRP-2, a parasite protein to released by mature-iRBCs²⁷. The relative deficit in parasite biomass estimated from the circulating ring stages may be accounted for by the pool of undetected ring-iRBCs retained in the spleen. Not least, retention in the spleen of the less deformable ring-iRBCs fits with available data linking reduced RBCs' mechanical deformability and spleen clearance in patients with RBC genetic disorders. Indeed, LORCA-based measurements in spleen-intact and splenectomized thalassemic patients has provided the only approximation of the EI below which RBC retention occurs in the spleen, namely 0.45 at 30 Pascal³⁹⁰. This is remarkably close to the value estimated here for ring-iRBCs (0.47 at 30 Pascal).

Because the LORCA measures the EI of a population of RBCs, the EI distribution (uni- or multi-modal) of individual ring-RBCs and the extent of individual variation is unknown. Deformability studies using single-cell tools have documented the reduced deformability of ring-iRBCs compared to uninfected RBCs from the same culture^{15,19,28,31}, and furthermore evidenced wide variations between individual cells. Multiple parasite and/or host-cell factors could contribute to ring-iRBC heterogeneously reduced deformability. The erythrocyte population is known to be heterogeneous, circulating erythrocytes displaying different levels of maturation and senescence³². As for the parasite, there is increasing evidence for heterogeneous expression by the individual merozoites of an array of surface molecules implicated in ligand-receptor interactions and of molecules discharged at the time of RBC merozoite invasion^{18,33}. Some of these interact with the erythrocyte cytoskeleton and contribute to host-cell membrane remodelling in young stages and to erythrocyte membrane stiffening after invasion^{13,19}. Others, such as Ring Surface Protein-2²⁰ predominantly associate with the surface of uninfected RBCs¹⁸, making their direct involvement in ring-iRBC spleen retention unlikely.

What is the ultimate fate of ring-iRBCs retained in the spleen RP: multiplication or destruction? A full intra-splenic maturation process of ring-iRBCs leading to intra-splenic reinvasion could theoretically take place, although the microenvironment in the cords of the RP has been considered hostile to RBCs³⁴. It is possible that spleen-retained ring-IRBCs are eventually phagocytosed as innate phagocytosis of ring-iRBCs has been observed during static in vitro experiments³⁵. Ring-iRBC destruction in the spleen is predicted to impact on the in vivo multiplication factor per cycle (IMF), which interestingly was lower (namely 3-16) in patients as assessed by counting circulating ring-iRBCs^{36,37} than predicted based on theoretical reinvasion rates (i.e. 18-24)³⁶. Such a discrepancy, usually interpreted as inefficient invasion in vivo, can also be viewed as resulting from spleen retention/clearance of a fraction of ring-iRBCs. Whatever the fate of ring-iRBCs within the spleen, their local retention reduces the parasite biomass that will sequester a few hours latter in vital organs such as the brain or the lungs.

Furthermore, cryptic ring-iRBCs retention in the spleen may explain why, in acute malaria, only a low proportion of the observed total RBC loss could be attributed to cumulated loss of iRBCs³⁸, since this was based on counting circulating ring-iRBC, ignoring the spleen-retained pool of ring-iRBCs.

Because they both implicate deformability-sensing mechanisms, spleen handling/clearance of ring-iRBCs and that of uninfected RBCs are linked. A link between spleen RBC filtration and parasite multiplication rate has been speculated^{15,39,40} but—in humans infected with Plasmodium falciparum—the iRBC population submitted to the spleen filtration process remained unidentified. Indeed, while mature-iRBCs are known to escape spleen filtration through sequestration, circulating ring-iRBCs visualized in the peripheral blood of patients were not considered susceptible to spleen retention. Our discovery that, shortly after invasion, a subpopulation of Plasmodium falciparum ring-iRBC is susceptible to spleen retention adds new strength to this link between RBC filtration and parasite multiplication. We thus infer that acquired or hereditary shift in the RBC deformability, as well as acquired or hereditary variation of the threshold for RBC retention by the spleen potentially impact on the clinical outcome of Plasmodium falciparum infection. Low-level spleen retention of ring-iRBCs may result in rapidly rising parasite loads leading to cerebral malaria or multiorgan failure, before anaemia and splenomegaly could develop. In contrast, high-level spleen retention of both RBCs and ring-iRBCs may generate a subacute infection accompanied by intra-splenic hemolysis, and favour the occurrence of severe anemia, frequently associated with a palpable splenomegaly. This could explain why severe anemia and cerebral malaria rarely occur simultaneously in the same patient. This novel concept triggers a shift in malaria research fields, such as modelling of infection kinetics, estimation of parasite loads, adjuvant therapy targeting RBC deformability⁴¹, analysis of risk factors for severe clinical forms⁴² (including patient age), or study of innate protection linked to hemoglobin inherited disorders.

### Example B

### Example of a Method for Screening for Compounds Increasing Rigidity of Plasmodium falciparum Ring-Infected Red Blood Cells (Ring-iRBCs)

Parasite culture. The Palo-Alto strain of Plasmodium falciparum (FUP)⁴⁴ is used. Plasmodium falciparum ring-iRBCs are cultured as previously described⁴⁵. Briefly, parasites are synchronised by selecting the ring stages by successive treatments (at least two successive treatments) with 5% (w/v) sorbitol (5 minutes at 37° C.) in order to obtain their complete reinvasion in 4 hours. Ring-infected RBCs are then incubated at 37° C. in complete medium (RPMI 1640 medium, bicarbonate, 25 mM glutamine, 0.2% glucose, 100 μM hypoxanthine, 10 μg/ml gentamicine and 10% AB+inactivated human serum pool) containing 0±RBCs in 75 cm² flasks treated for 30 seconds with an atmosphere of 1% O₂, 3% CO₂ and 96% N₂.

In vitro test. Schizonts (44 to 48H) are enriched by suspension in a gelatinous solution (Plasmion®). They are then allowed to re-invade fresh O+ RBCs (i.e. O+ RBCs of less than 8 days or of 8 days of age, preferably O+ RBCs of less than 8 days of age) in complete medium at 37° C. Ten hours after the onset of incubation (age of 80-90% of the ring-stage RBCs), a compound to be tested is added to the culture, which is then further incubated for 2 hours at 37° C. After removal of the culture supernatant (by centrifugation at 1500 rounds per minute for 5 minutes), the pellet is resuspended in PBS (hematocrit level of 50%) and used to analyse deformability of ring-iRBCs.

Analysis of Ring-iRBCs Deformability

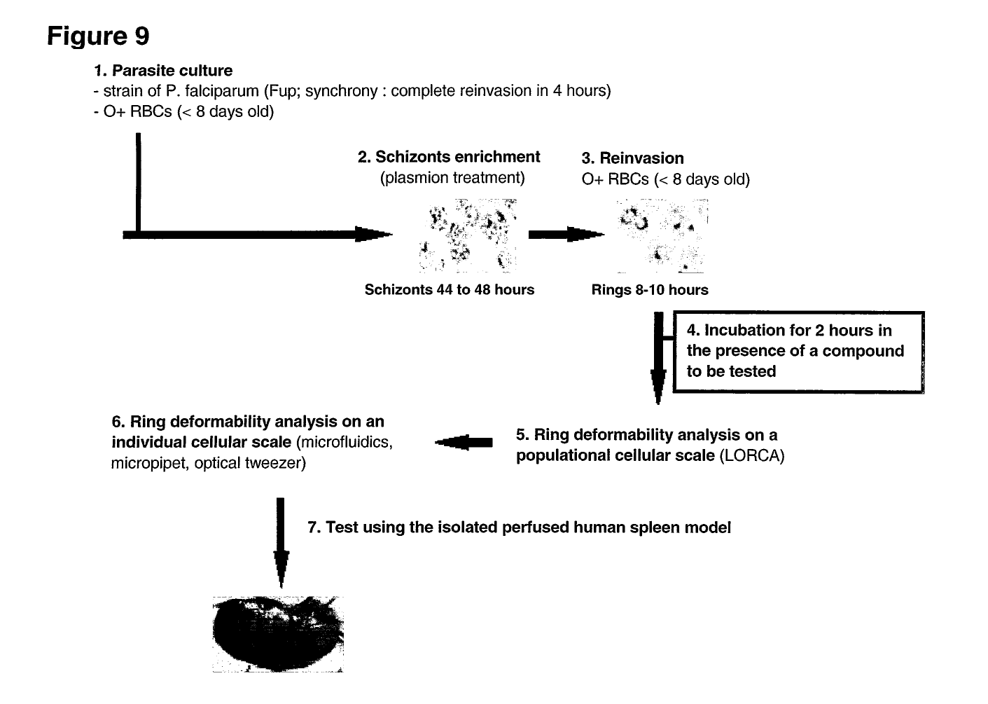

Deformability assessment is performed in several steps (see FIG. 9):

1. Use of a LORCA with a screening threshold for the decrease of the mean EI of 10%.

2. Analysis of the compounds identified by the LORCA technique as being able to increase rigidity of ring-iRBCs, by means of more accurate techniques, which enable to assess deformability of ring-iRBCs at an individual cellular scale (with a LORCA, analysis is performed at a populational cellular scale), e.g., by means of micropipets, optical tweezers, or a microfluidic device. These techniques allow to evaluate more precisely EI dispersion and thus the percentage of ring-iRBCs that could be retained in the spleen after exposure to a screened compound.

3. Optionally, few candidate compounds are tested using an isolated perfused human spleen model.

The LORCA technique is performed as previously described⁴⁶. The principle of this method consists in submitting, to a shear force and to a constant temperature, a population of iRBCs and a population of uninfected RBCs, said populations being suspended in a viscous solution (5% polyvinylpyrrolidone in PBS, viscosity=30 mPa·sec at 37° C.) and being introduced in a slit located between two concentric cylinders. The shear force resulting from the rotation of one of the cylinders (the outer one) leads to an elongation of the RBCs in the form of an ellipse. A laser beam emitted from a laser diode traverses the RBCs or iRBCs suspension and is diffracted by these cells in the volume. The laser beam diffraction pattern that is projected on a projection screen is captured by a digital video camera and transmitted to a computer. A software then converts the ellipsoid diffraction pattern into a digit value: the elongation index (EI). Thus, the deformability unit is defined as the ratio of height of the ellipse to [height+width of the ellipse] 46, 47. This value is is the mean IE value of the RBCs population (infected RBCs population or uninfected RBCs population) contained in the suspension. Deformability of infected RBCs and of uninfected RBCs is evaluated over a range of shear forces (0.3 to 30 Pascal), which include 1.7 Pa (shear force in the microvessels of the deep tissue) and 30 Pa (shear force in red pulp sinuses). Using one LORCA, one technician is able to test 30 compounds per days, i.e. at least 100 compounds per week, if one takes into account the steps of parasites preparation and storage of the generated data.

### Example C

### Filtration of RBCs and Plasmodium falciparum-Infected RBCs (Pf-iRBCs) as a Surrogate for Spleen Filtering Function

### Material and Methods

Parasitized RBCs: clinical samples and culture. P. falciparum (D10, 3D7 or FUP/Palo Alto strain) was cultured in vitro as described⁶¹. RBCs were washed and stored at 4-8° C. for 1-10 days. Pf-iRBCs present in the blood of patients were stored for less than 24 hours at 4-8° C. after collection.

Source and storage of normal RBCs. RBCs were from healthy blood donors (blood bank, Rungis, France). Leucocytes were removed by centrifugation. RBCs were stored at 4° C. and used after less than 8 days of storage.

Filtration with Channel-Perforated Membranes

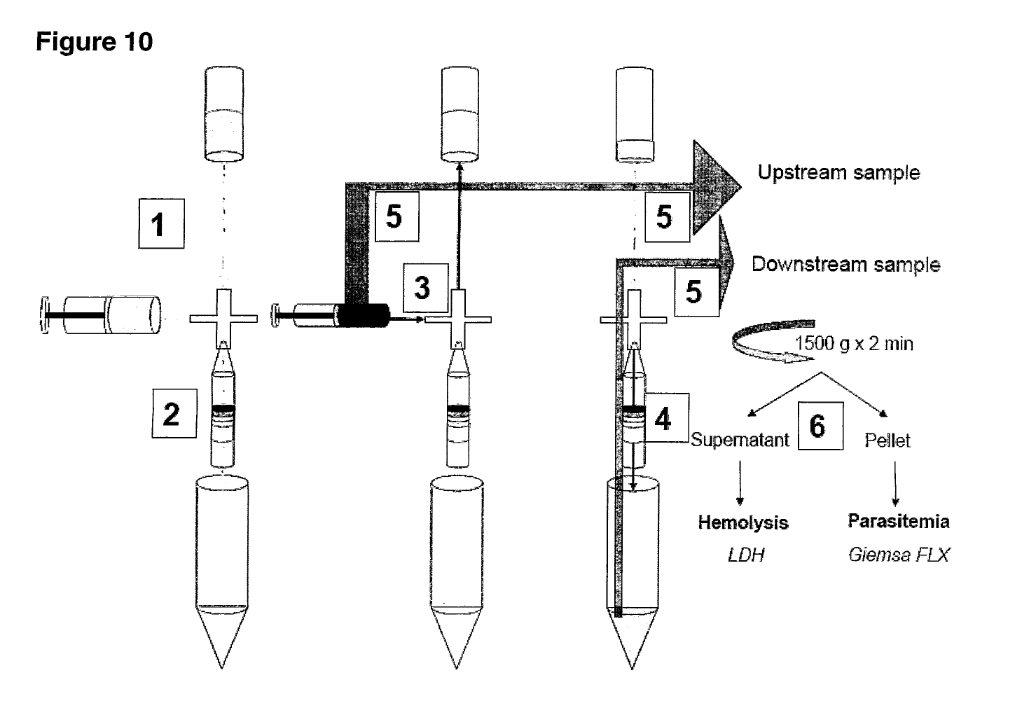

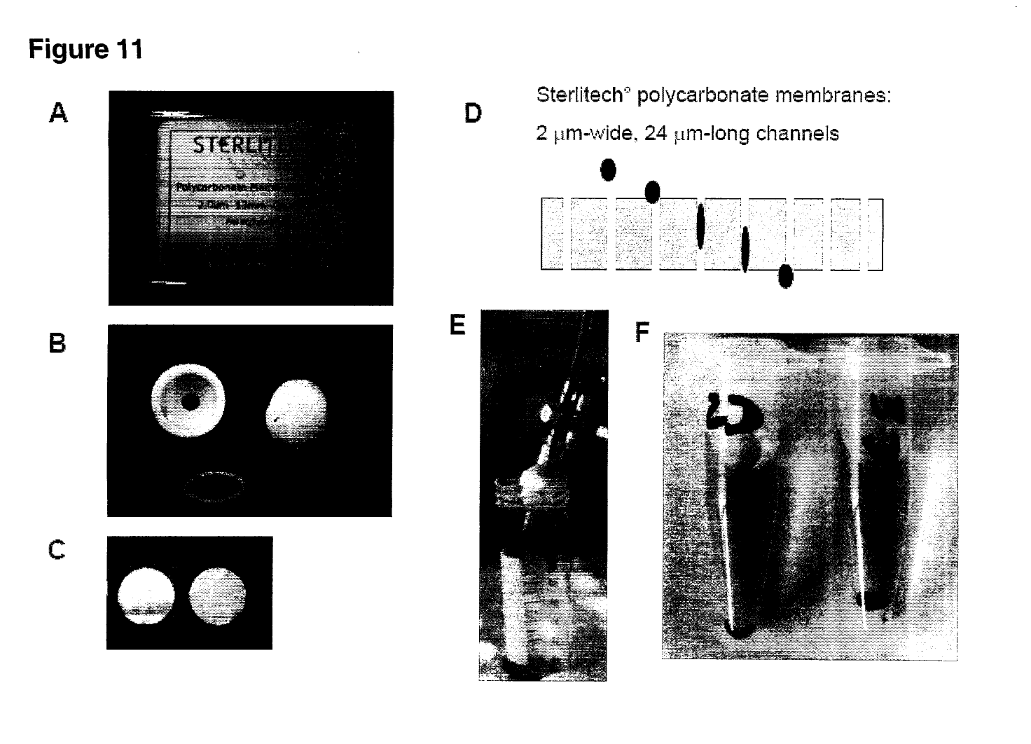

Column surfaces and membranes were blocked with suspending medium (RPMI+4% albumin+5% Plasmion®) during 15 minutes prior to introduction of Pf-iRBCs. Cultured or “clinical” Pf-iRBCs were allowed to flow through 24 μm-thick polycarbonate membranes perforated with 0.8-8 μm-wide channels (Sterlitech Corporation; see FIGS. 10 and 11), after suspension at 2%-2.5% hematocrit in RPMI supplemented with 4% albumin and 5% Plasmion®. Filtration was performed at 34-37° C. under a constant pressure (80-85 cm of water). Preliminary experiments showed that flow was unimpaired when the channel diameter was ≧3 μm whereas no flow was observed through pores the diameter values of which are ≦1 μm. Subsequent experiments were done with 2 μm-wide channels. No retention of Pf-iRBCs was observed when channel width was ≧3 μm. Outflow from 2 μm-wide channels membranes was 0.1-1 ml/min.

“Upstream” and “downstream” RBC sub-populations were retrieved (as illustrated on FIG. 10) and centrifuged (2 minutes at 1500 g) and the obtained RBC pellets were used for quantification.

Tin Bead Filtration

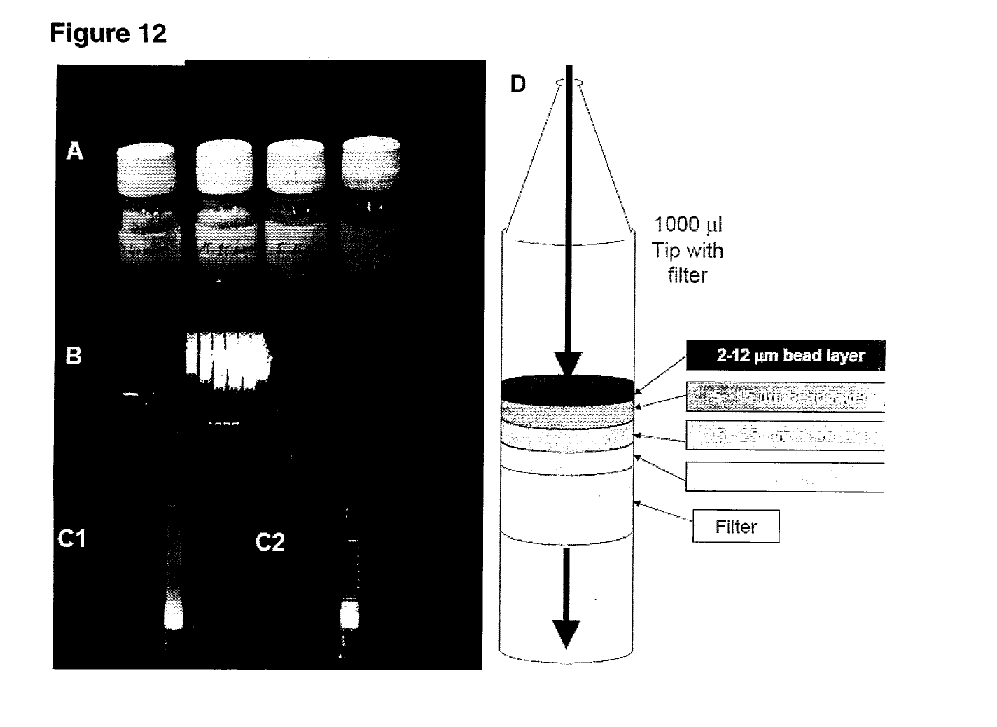

Cultured or “clinical” Pf-iRBCs were allowed to flow through 0.5-2 mm-thick layers of tin beads (Industrie des poudres sphériques (IPS), Annemasse, France) of increasing diameter (from 2-12 μm, 5-15 μm, 15-25 μm and more than 40 μm; see FIGS. 12 and 13) after suspension at 2-2.5% hematocrit in PBS or RPMI supplemented with 1% albumax II® (Gibco). Filtration was performed at 20-25° C. under a constant pressure (80-85 cm of water). Column surfaces and bead layers were blocked with suspending medium (PBS+1% Albumax II®) during 15 minutes prior to introduction of Pf-iRBCs. Preliminary experiments showed no retention of Pf-iRBC with thin bead layers made of beads of diameter 5-15 or 15-25 or more than 40 μm, or with only the filter in the tip used to maintain the bead layers. Further experiments showed that a mixture of equal weight of 5-15 μm and 15-25 μm (thereafter referred to as “5-25 μm layer”) induced the retention of iRBCs provided that the thickness of the layer was >5 mm. The filtration protocol was then modified by the use of a 7 mm-thick layer of beads of diameter 5-25 μm.

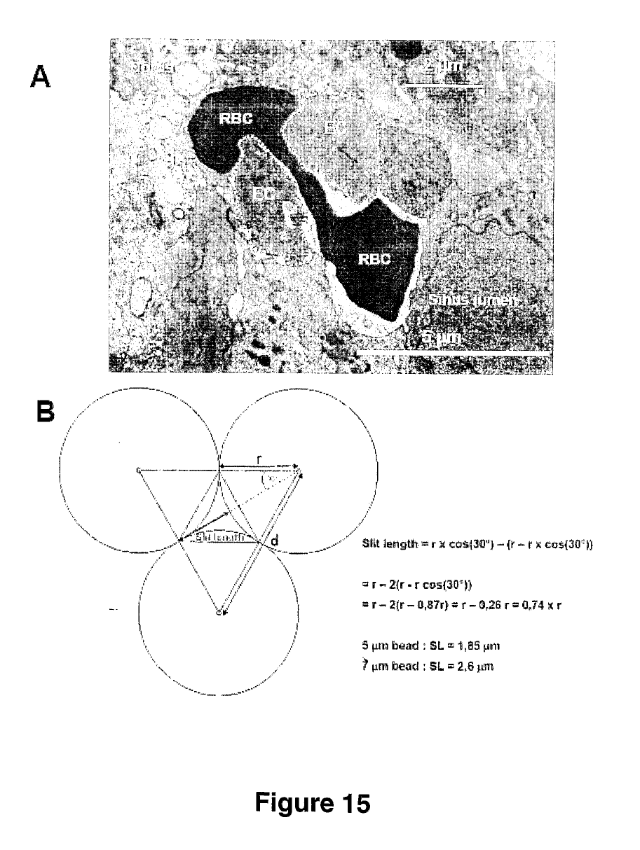

Using beads of diameter 5-25 μm, channel width (i.e., inter-bead space) varies between 1.85 μm (width of the channel formed by three beads of diameter 5 μm placed side by side) and 9.4 μm (width of the channel formed by three beads of diameter 25 μm placed side by side). The width of the channel formed by three beads of diameter 7 μm placed side by side is 2.6 μm (see FIG. 15B). If each class of beads of different diameter is equally represented, beads of diameter 5-7 μm account for 10% of the beads that are used. Therefore, (0.1)³=0.1% of channels will be 1.85-2.6 μm-wide. Also, the probability is low to create channels that are both long (>4 μm) and narrow (<2.6 μm).

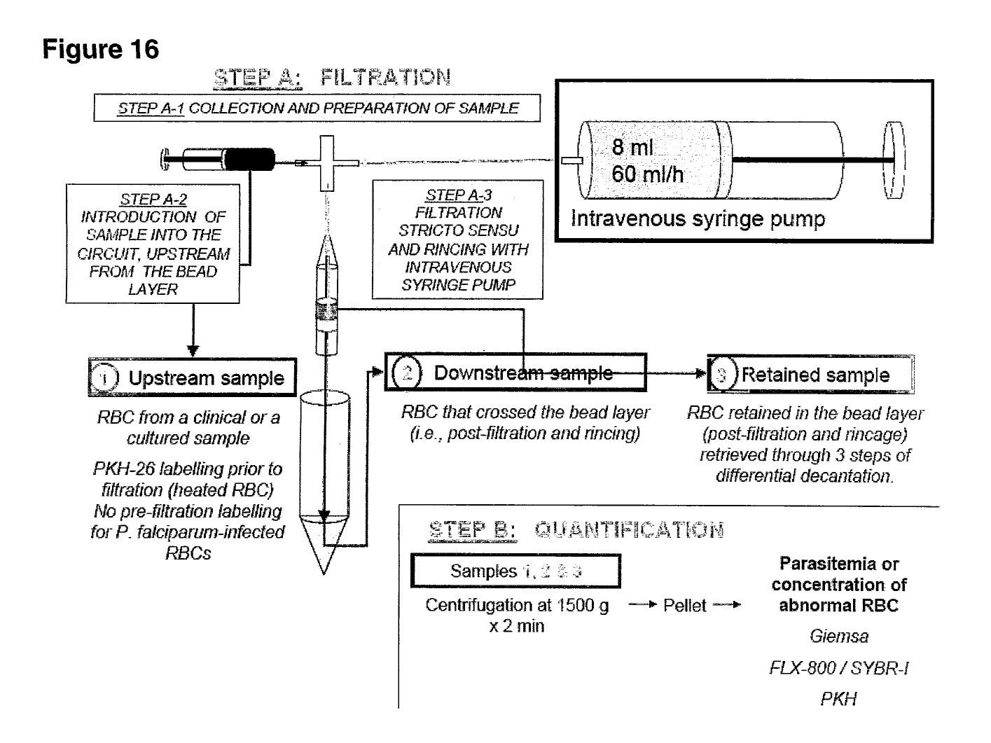

600 μl of the 2-2.5% hematocrit sample (corresponding to a 12-15 μl pellet) were introduced into the hermetic circuit upstream from the bead layer through a 3-way stopcock (see FIG. 16). An electric pump was then used to generate a constant 1 ml/min flow of PBS-Albumax II through the layer during 8 minutes (8 ml final volume). Upper pressure limit was 999 mbars.

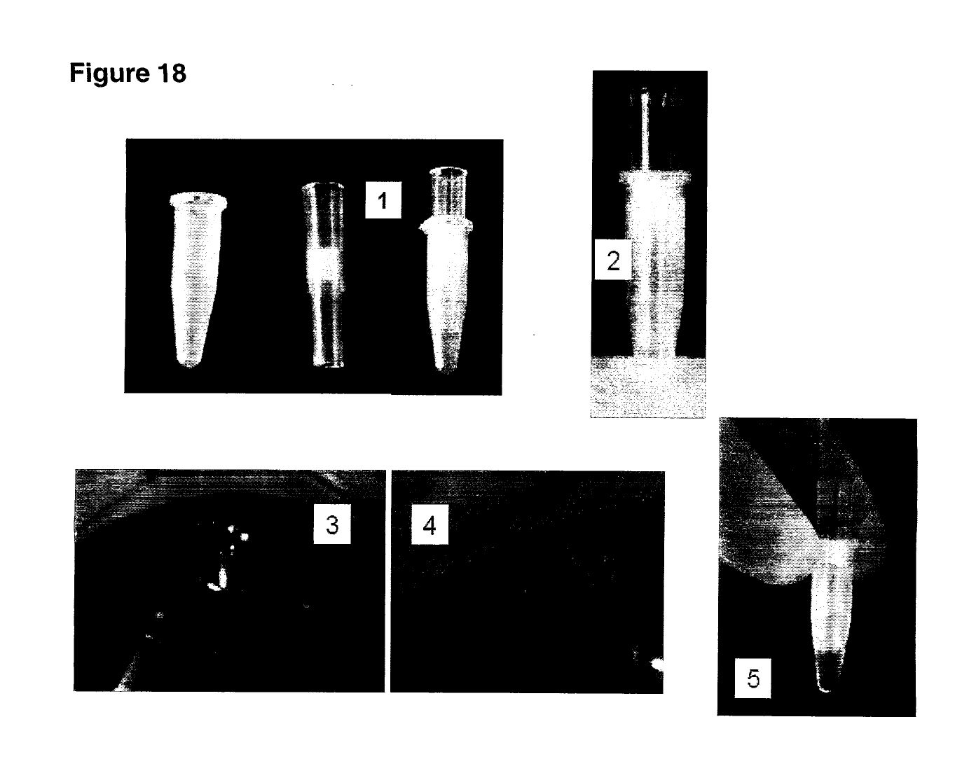

Centrifugation-based filtration. Alternatively, bead-containing tips used as filtering units were adjusted to 1.5 to 2 ml Eppendorf tubes (see FIG. 18) then centrifuged for 1-2 minutes at 1500-2500 (until the whole sample had flown through). The bead layer(s) was (were) rinced twice with 600-800 μl of suspending medium by the same centrifugation method. Upstream, downstream and retained RBC sub-populations were retrieved and processed for quantification as described below.

Retrieval of “upstream”, “downstream” and “retained” RBC sub-populations:

At least 300 μl of the “upstream” sample were reserved prior to filtration, centrifuged (5 min at 1500 g), and the obtained RBC pellet was used for quantification;

The 8 ml of “downstream” sample containing RBCs that had flown through the bead layer were centrifuged for 5 min at 1500 g, and the thus obtained 10-14 μl RBC pellet was used for quantification.

The bead layer was retrieved in 1.5 ml Eppendorf tubes at the end of the filtration process. Three steps of 1-3 minutes decantation by gravity allowed for the retrieval of a 1-5 μl RBC pellet containing less than 3% beads.

Control of Haemolysis. In selected experiments, human lactate dehydrogenase (LDH) concentration was quantified in the supernatant from centrifuged “upstream”, “downstream” and “retained” samples for the determination of haemolysis. A similar volume of suspending medium in which RBCs at 2-2.5% hematocrit had been haemolysed with 0.2% saponin was used as a 100% haemolysis control.

Quantification of Plasmodium-Hosting RBCs or Abnormal RBCs in Samples:

Giemsa staining: Methanol-fixed thin smears were stained with 10% Giemsa for 10 minutes, and the proportion of parasitized RBCs counted against 2000 RBCs. Results were expressed as a stage-specific parasitaemia.

Fluorescence-based quantification of abnormal (heated) RBCs. RBCs were stained with PKH-26 (Red Fluorescent Cell Linker Kit; Sigma) prior to heating for 5-20 minutes at 50° C. Quantification was performed by counting the proportion of PKH-positive RBCs on blinded pictures.

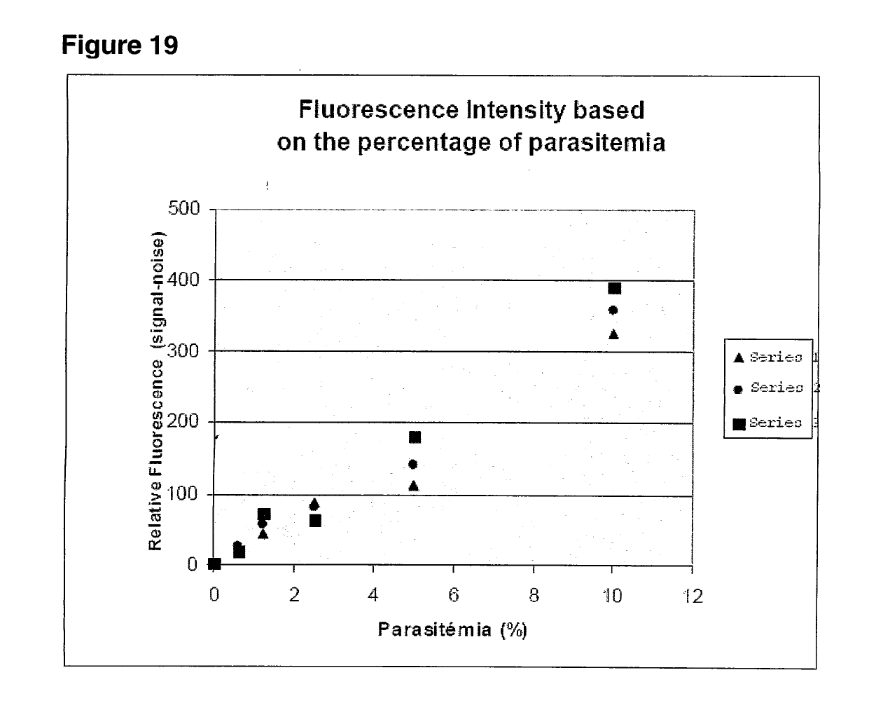

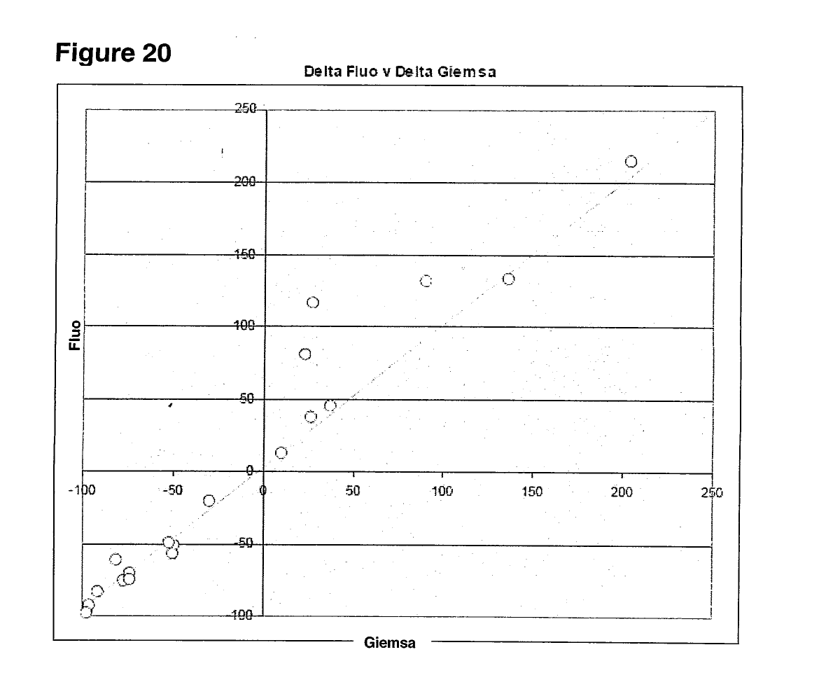

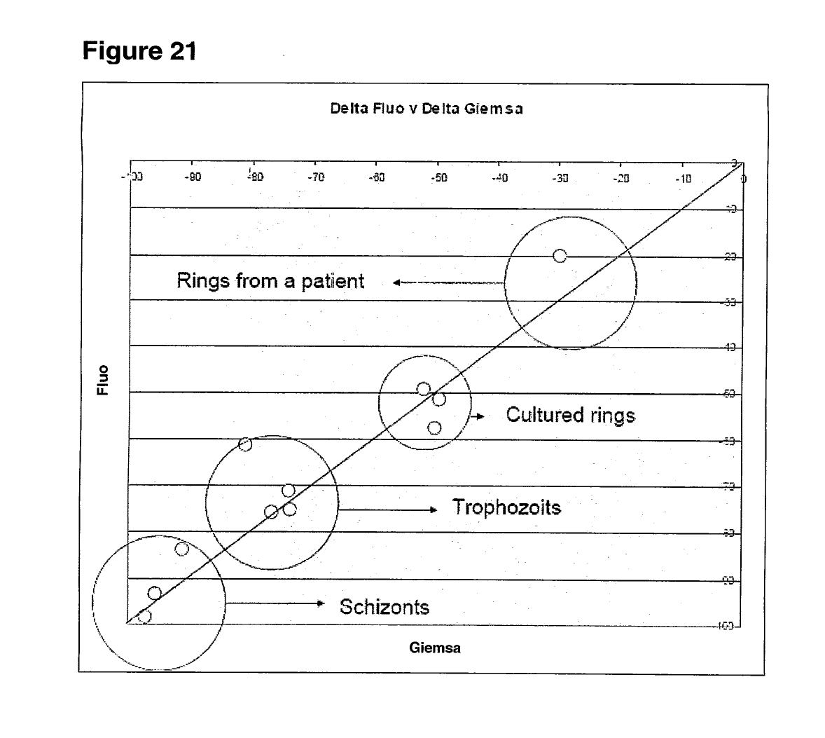

Fluorescence-based quantification of parasitized RBCs. In order to express results as a concentration of parasitized RBCs (thereby equivalent to parasitaemia on Giemsa-stained smears), triplicate 4 μl samples of pellet (from either upstream, downstream, or retained samples) were carefully aspirated and diluted in lysis buffer (Tris 20 mM (pH 7.5) EDTA 5 mM, saponin 0.008%, Triton-X100 0.08%) containing 0.1% of SYBR-I (SYBR green I: Molecular Probes, Invitrogen, Carlsbad, Calif.). Fluorescence intensity was quantified with a FLX-800 counter (FLX-800 microplate fluorescence reader; Biotek instruments, Winooski, Vt., USA) after 30 minutes of incubation at room temperature. Preliminary experiments showed a linear correlation between parasitaemia and fluorescence intensity (see FIG. 19). This new fluorescence method provided results similar to that of the reference Giemsa quantification method to estimate the decrease (or increase) of parasitized RBC concentration from upstream to downstream (or from upstream to the bead is layer) (see FIGS. 20 and 21).

### Results

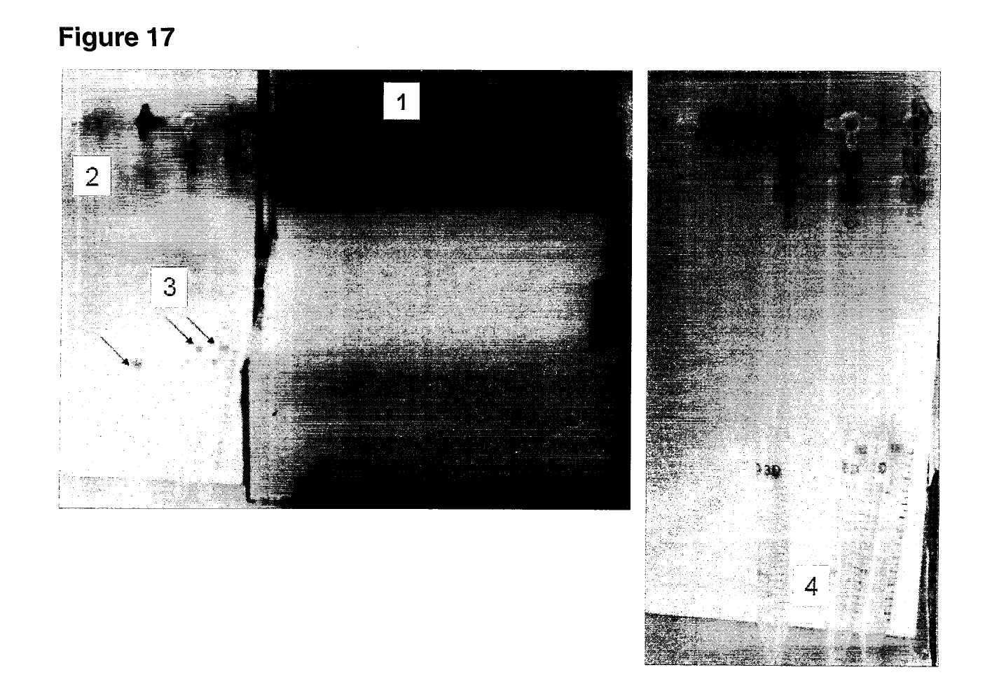

Two different filtration techniques were used (see FIGS. 10-18):

gravity-driven filtration with channel-perforated membranes; and

bead layer filtration.

Experimental filtration was associated with a significant retention of Pf-iRBCs (see Table II).

Filtration-associated retention of Pf-iRBCs displayed features strongly reminiscent of retention in both isolated-perfused human spleens and patient spleens. We indeed observed:

(i) complete/almost complete retention of trophozoites/schizonts (mature forms)—hosting RBCs (see Table II and FIG. 21);

(ii) partial retention of ring-hosting RBCs (see Table II and FIG. 21);

(iii) no or partial retention of mature gametocyte-hosting RBCs (see Table II);

(iv) greater retention of ring-hosting RBCs from in vitro culture as compared to ring-hosting RBCs from the peripheral blood of patients (ring-hosting RBCs present in patient blood either (i) have not been submitted to the “IES challenge” yet (on average a RBC crosses an IES once every 2-3 hours⁵⁴) or (ii) were deformable/filterable enough to keep circulating despite having been submitted to this challenge) (see Table II and FIG. 21); and that

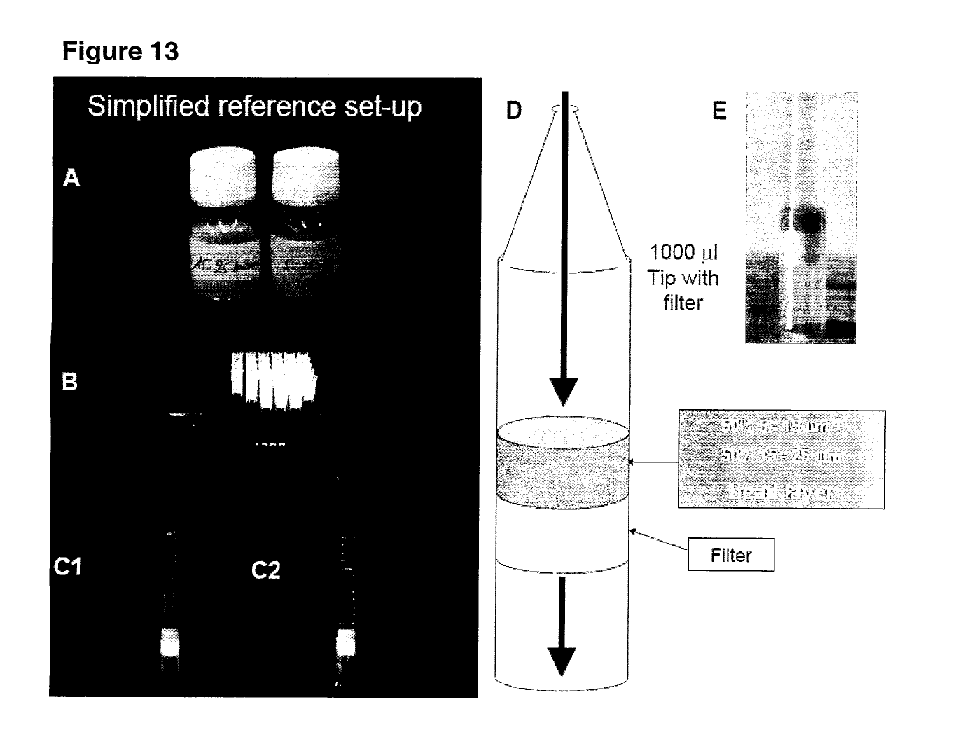

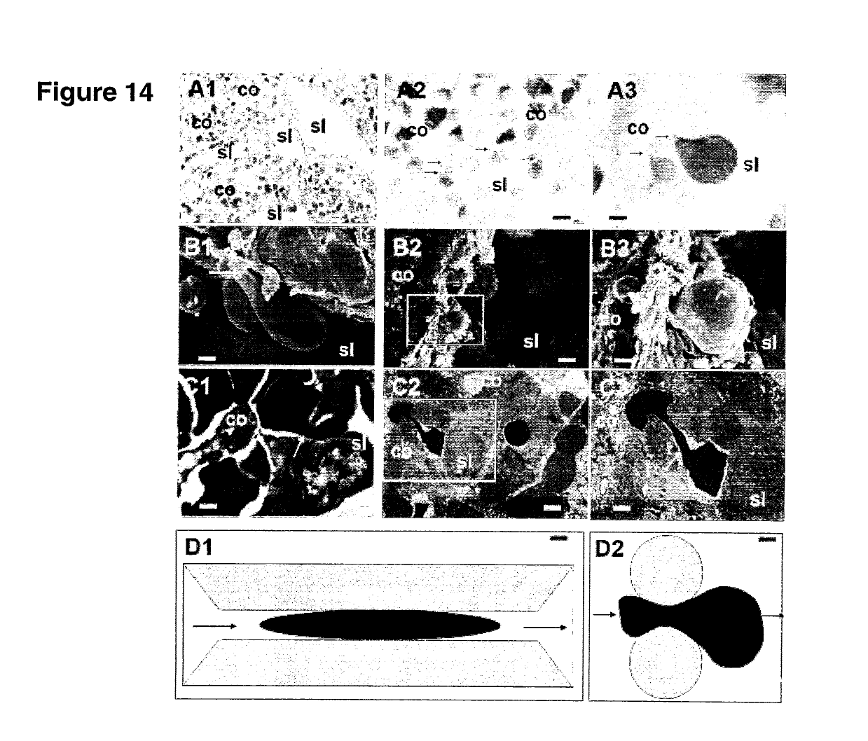

(v) bead layers—the geometry of which mimics the spleen red pulp circulatory beds better than does that of channel-perforated membranes (see FIGS. 14 and 15)—displayed retention features that better mimic spleen filtration, specifically the absence of haemolysis (see Table II) and a better preservation of RBC morphology.

Experimental filtration through bead layers was associated with a significant retention (total retention) of heated RBCs (see Table II), showing that potential applications of the method expand across several RBC abnormalities.

Experimental filtration of RBCs and Pf-iRBCs according to the method of the invention (in particular as illustrated on FIGS. 10-18) is the best available surrogate for physiological/pathophysiological spleen filtration.

RBCs retained in the bead layers can be retrieved at the end of the filtration process by a very simple differential decantation method, opening the way for the molecular analysis of RBC sub-populations of defined phenotypes.

The two stages of Pf-hosting RBCs that circulate in the peripheral blood of patients, namely ring-hosting RBCs and mature gametocytes-hosting RBCs, are potentially submitted to spleen retention. Drugs reducing the deformability of circulating Pf-iRBCs should therefore trigger/increase their retention in the spleen.

A high-throughput screening method is the way toward the discovery of active compounds specifically decreasing the deformability of Pf-iRBCs, and experimental filtration provides a way to set-up this middle/high-throughput screening system. Activity will be defined as the ability of a compound to induce/increase filtration-mediated Pf-iRBC retention (decreased filterability of Pf-iRBCs). The 3 essential screening steps, i.e., (i) exposure to compounds, (ii) filtration, and (iii) counting of parasite densities before and after filtration, are adaptable to middle to high-throughput screening constraints.

TABLE II
Mean (SD; range) of the retention rate stated in percentages of Pf-iRBCs in filters
(Channel-perforated membranes or tin bead layers).

| | | Tin bead layers (5-25 μm) |
|---|---|---|
| | Channel-perforated | Using the reference syringe-based |
| Source of Pf-iRBC | membranes (Sterlitech°) | method |
| Ring-hosting RBCs in | −43.5% (9.9; −33% to −62%) | −19% (16; +5% to −30%) |
| patient blood | (8 experiments) | (4 experiments) |
| Gametocytes-hosting | +72% (1 experiment) * | ND |
| RBCs in patient blood | | |
| Haemolysis | 24.3% (LDH method) | LDH not done |
| (ring-hosting RBCs) | (2 experiments) | No observable haemolysis |
| Ring-hosting RBCs in | −69% (10.5; −56% to −83%) | −42% (9.01; −30% to −59%) |
| culture | (5 experiments) | (7 experiments) |
| Trophozoites/Schizonts | −79% (18.1; −63% to −100%) | −86% (12.9; −63% to −100%) |
| in culture | (3 experiments) | (9 experiments) |
| Gametocytes-hosting | ND | −22% |
| RBCs in culture | | (1 experiment) |
| Haemolysis | Not measured but light red | <3% (LDH method) |
| | supernatant | (4 experiments) |
| Heated RBCs (50° C. × 5 − | ND | −100% |
| 20 minutes) | | (4 experiments) |

The retention rate stated in percentages was calculated using the following formula: [(parasitemia downstream from the filtering unit − parasitemia upstream from the filtering unit)/parasitemia upstream from the filtering unit] × 100.
* Paradoxal enrichment of gametocytes-hosting RBC is likely linked to the preserved deformability of mature gametocytes-hosting RBC and to the decreased deformability of a portion of uninfected RBC in the peripheral blood of patient.

Filtration by short centrifugation (1-2 minutes×1500-2500 g; see FIG. 18) induced retention rates of Pf-hosting RBCS similar to that obtained by the reference syringe-pressured flow filtration in a hermetic circuit (see Table III). This result opens the way to the automation of this experimental step.

Retention rates determined by a liquid-phase fluorescence quantification method, based on SYBR-I staining of parasite nucleic acids⁵⁶⁻⁶⁰, correlate with retention rates determined by the reference quantification of parasite densities by counting on Giemsa-stained smears (see FIGS. 19 and 20). This fluorescence-based quantification method of parasite densities upstream and downstream from filters (e.g., channel-perforated membranes or bead layers) opens the way to the automation of this experimental step.

TABLE III
Mean (SD; range) of the retention rate (stated in percentages) of Pf-iRBCs
retained in tin bead layers (using the centrifugation-based filtration method).

| | Tin bead layers (5-25 μm) |
|---|---|
| Source of Pf-iRBCs | Centrifugation-based filtration method |
| Ring-hosting RBCs in patient blood | −20% (1 experiment) |
| Ring-hosting RBCs in culture | −47% (−36%, −41%, −64%) |
| | (3 experiments) |
| Hemolysis | LDH not done |
| | No observable haemolysis |

The retention rate was calculated using the following formula: [(parasitemia downstream from the filtering unit − parasitemia upstream from the filtering unit)/parasitemia upstream from the filtering unit] × 100.

In a particular embodiment, the method of screening of the invention using the filtration method disclosed herein should display the following characteristics:

a. the filtering unit should have narrow (0.2-2 μm) and short (preferably <5 μm) pores;

b. read-out should be based on the variation of the concentration of red blood cells under test contained in the sample (e.g., Pf-infected red blood cells at different stages);

c. small volume (1-100 microliters of packed red blood cells) samples to should be used;

d. flow through the filtering unit should be gravity-driven or performed by flush or by aspiration or by centrifugation or by any method allowing the sample to flow through the filter, preferably at a low hematocrit (<5%);

e. screening should be performed according to a 3-main step process:
cultured ring- or mature gametocyte-hosting RBCs are exposed to known or new chemical entities from a library in a microplate;
a proportion of the volume of drug-exposed RBCs is aspirated through a filtering unit and the downstream sample is deposited in another microplate;
quantification of parasite concentrations in upstream and downstream plates is performed in parallel by an automated method. The result is expressed as the downstream/upstream concentration ratio. A compound inducing a significant decrease of this ratio as compared to the internal unexposed control is then considered as a hit;

f. although the nature of the filtering unit may be modified, its structure should be amenable to the retrieval of 1 μl to 1 ml of retained RBCs.

Discussion

Anopheles sp. may act as hosts and vectors of Plasmodium sp. only if mature male and female gametocyte-hosting RBCs were present in the blood meal. In areas where transmission is low together with other methods and tools, the curative anti-malarial therapies (artemisinin-based combination therapies, ACT) have been shown to result in reduced blood infectivity. ACTs reduce the number of parasites that produce gametocytes, and directly kill immature non-circulating gametocytes. However, ACTs are inactive on mature circulating gametocytes¹. In highly endemic areas, many human subjects carry mature gametocytes but have no symptom: because these subjects are not treated, the impact of ACT on transmission is limited. Primaquine is the only available anti-malarial drug active on mature gametocytes. Although mass administration of primaquine has been successful in selected areas, its administration schedule and its toxicity in G6PD-deficient subjects make it unsuitable for large scale use in highly endemic areas of Africa'. It is therefore crucial to develop new drugs that would be active on mature gametocytes, suitable for mass administration.

A compound inducing the spleen-dependent clearance of mature gametocytes-hosting RBCs from the circulating blood would be candidate for development as a transmission-blocking drug. This drug would indeed make gametocytes unavailable to Anopheles sp. thereby removing them from the transmission cycle.

A drug inducing the spleen-dependent clearance of ring-hosting RBCs from the circulating blood would be a fast-acting drug, useful to treat clinical attacks and prevent evolution toward severe manifestations of the disease. Removal of Pf-iRBCs at the ring stage (i.e., before they mature to the Pf-EMP1-expressing trophozoite stage) would prevent them from sequestering in arterioles, capillaries and venules. Sequestration of trophozoite- and schizont-harbouring RBCs is strongly associated with severe clinical manifestations of P. falciparum infection in humans.

A drug displaying both above-mentioned properties might have a major impact.

Experimental filtration of drug-exposed Pf-iRBCs either collected after a few hours of therapy of after in vitro exposure to drugs may be a basis for a phenotypic determination of drug sensitivity. Accelerated clearance of Pf-iRBCs exposed to existing anti-malarial drugs (e.g., artemisinin derivatives) may be due in part to their deformability-dependent retention or destruction (e.g., through pitting) in the spleen.

As it allows the concentration of ring-hosting RBCs, experimental filtration as disclosed herein may provide cheap, user-friendly tools useful for the diagnosis of malaria, as well as for experimental studies.

In a wider perspective, experimental filtration may facilitate the diagnosis or prognosis estimation of inherited RBC disorders that affect RBC deformability especially when genotype/phenotype correlation fails to do so.

Because experimental RBC filtration is the best available surrogate for spleen filtering function, it may provide the basis for a useful test to estimate spleen function in patients with inherited or acquired immune dysfunctions as well as inherited or acquired RBC disorders.

Because stiff RBCs can be retrieved from the bead layers, comparative analysis at the cellular, sub-cellular and molecular level can be performed with the different subpopulations available (upstream=“parental”, down stream=“deformable” and retained in the bead layer=“non deformable”). This new filtration method is also amenable to automation and allows the clearance or concentration of stiff RBCs, with wide experimental and medical applications in inherited or acquired RBC disorders (including malaria).

### REFERENCES

1. Maitland K, Marsh K. Pathophysiology of severe malaria in children. Acta Trop. 2004; 90:131-140.
2. Wyler D J, Gallin J I. Spleen-derived mononuclear cell chemotactic factor in malaria infections: a possible mechanism for splenic macrophage accumulation. J. Immunol. 1977; 118:478-484.
3. David P H, Hommel M, Miller L H, Udeinya I J, Oligino L D. Parasite sequestration in Plasmodium falciparum malaria: spleen and antibody modulation of cytoadherence of infected erythrocytes. Proc Natl Acad Sci USA. 1983; 80:5075-5079.
4. Boone K E, Watters D A. The incidence of malaria after splenectomy in Papua New Guinea. Bmj. 1995; 311:1273.
5. Bach O, Baier M, Pullwitt A, et al. Falciparum malaria after splenectomy: a prospective controlled study of 33 previously splenectomized Malawian adults. Trans R Soc Trop Med. Hyg. 2005; 99:861-867.
6. Davidson R N, Wall R A. Prevention and management of infections in patients is without a spleen. Clin Microbiol Infect. 2001; 7:657-660.
7. Buffet P A, Milon G, Brousse V, et al. Ex vivo perfusion of human spleens maintains clearing and processing functions. Blood. 2006; 107:3745-3752.
8. Looareesuwan S, Merry A H, Phillips R E, et al. Reduced erythrocyte survival following clearance of malarial parasitaemia in That patients. Br J Haematol. 1987; 67:473-478.
9. Looareesuwan S, Davis T M, Pukrittayakamee S, et al. Erythrocyte survival in severe falciparum malaria. Acta Trop. 1991; 48:263-270.
10. Urban B C, Hien T T, Day N P, et al. Fatal Plasmodium falciparum malaria causes specific patterns of splenic architectural disorganization. Infect Immun. 2005; 73:1986-1994.
11. Groom A C, Schmidt E E, MacDonald I C. Microcirculatory pathways and blood flow in spleen: new insights from washout kinetics, corrosion casts, and quantitative intravital videomicroscopy. Scanning Microsc. 1991; 5:159-173; discussion 173-154.
12. Weiss L. Mechanisms of splenic control of murine malaria: cellular reactions of the spleen in lethal (strain 17XL) Plasmodium yoelii malaria in BALB/c mice, and the consequences of pre-infective splenectomy. Am J Trop Med. Hyg. 1989; 41:144-160.
13. Silva M D, Cooke B M, Guillotte M, et al. A role for the Plasmodium falciparum RESA protein in resistance against heat shock demonstrated using gene disruption. Mol. Microbiol. 2005; 56:990-1003.
14. Deitsch K W, Wellems T E. Membrane modifications in erythrocytes parasitized by Plasmodium falciparum. Mol Biochem Parasitol. 1996; 76:1-10.
15. Cranston H A, Boylan C W, Carroll G L, et al. Plasmodium falciparum maturation abolishes physiologic red cell deformability. Science. 1984; 223:400-403.
16. Chen L T. Microcirculation of the spleen: and open or closed circulation? Science. 1978; 201:157-159.
17. Correas J M, Kurtisovski E, Bridal S L, Amararene A, Helenon O, Berger G. Optimizing an ultrasound contrast agent's stability using in vitro attenuation measurements. Invest Radiol. 2002; 37:672-679.
18. Layez C, Nogueira P, Combes V, et al. Plasmodium falciparum rhoptry protein RSP2 triggers destruction of the erythroid lineage. Blood. 2005; 106:3632-3638.
19. Mills J P, Diez-Silva M, Quinn D J, et al. Effect of plasmodial RESA protein on deformability of human red blood cells harboring Plasmodium falciparum. Proc Natl Acad Sci USA. 2007; 104:9213-9217.
20. Pouvelle B, Buffet P A, Lepolard C, Scherf A, Gysin J. Cytoadhesion of Plasmodium falciparum ring-stage-infected erythrocytes. Nat. Med. 2000; 6:1264-1268.
21. Hardeman M R, Ince C. Clinical potential of in vitro measured red cell deformability, a myth? Clin Hemorheol Microcirc. 1999; 21:277-284.
22. Buffet P A, Gamain B, Scheidig C, et al. Plasmodium falciparum domain mediating adhesion to chondroitin sulfate A: a receptor for human placental infection. Proc Natl Acad Sci USA. 1999; 96:12743-12748.
23. Chen L T, Weiss L. Electron microscopy of the red pulp of human spleen. Am J Anat. 1972; 134:425-457.
24. Dondorp A M, Kager P A, Vreeken J, White N J. Abnormal blood flow and red blood cell deformability in severe malaria. Parasitol Today. 2000; 16:228-232.
25. Weiss L, Chen L T. The differentiation of white pulp and red pulp in the spleen of human fetuses (72-145 mm crown-rump length). Am J Anat. 1974; 141:393-413.
26. Silamut K, Phu N H, Whitty C, et al. A quantitative analysis of the microvascular sequestration of malaria parasites in the human brain. Am J Pathol. 1999; 155:395-410.
27. Dondorp A M, Desakorn V, Pongtavornpinyo W, et al. Estimation of the total parasite biomass in acute falciparum malaria from plasma PfHRP2. PLoS Med. 2005; 2:e204.
28. Nash G B, O'Brien E, Gordon-Smith E C, Dormandy J A. Abnormalities in the mechanical properties of red blood cells caused by Plasmodium falciparum. Blood. 1989; 74:855-861.
29. Prommano O, Chaisri U, Turner G D, et al. A quantitative ultra-structural study of the liver and the spleen in fatal falciparum malaria. Southeast Asian J Trop Med Public Health. 2005; 36:1359-1370.
30. Dondorp A M, Chotivanich K T, Fucharoen S, et al. Red cell deformability, splenic function and anemia in thalassaemia. Br J Haematol. 1999; 105:505-508.
31. Li J, Dao M, Lim C T, Suresh S. Spectrin-Level Modeling of the Cytoskeleton and Optical Tweezers Stretching of the Erythrocyte. Biophys J. 2005.
32. Lutz HU. Innate immune and non-immune mediators of erythrocyte clearance. Cell Mol Biol (Noisy-le-grand). 2004; 50:107-116.
33. Cortes A, Benet A, Cooke B M, Barnwell J W, Reeder J C. Ability of Plasmodium falciparum to invade Southeast Asian ovalocytes varies between parasite lines. Blood. 2004; 104:2961-2966.
34. Levesque M J, Groom A C. pH environmental of red cells in the spleen. Am J. Physiol. 1976; 231:1672-1678.
35. Ayi K, Turrini F, Piga A, Arese P. Enhanced phagocytosis of ring-parasitized mutant erythrocytes: a common mechanism that may explain protection against falciparum malaria in sickle trait and beta-thalassemia trait. Blood. 2004; 104:3364-3371.
36. White N J, Chapman D, Watt G. The effects of multiplication and synchronicity on the vascular distribution of parasites in falciparum malaria. Trans R Soc Trop Med. Hyg. 1992; 86:590-597.
37. Simpson J A, Aarons L, Collins W E, Jeffery G M, White N J. Population dynamics of untreated Plasmodium falciparum malaria within the adult human host during the expansion phase of the infection. Parasitology. 2002; 124:247-263.
38. Price R N, Simpson J A, Nosten F, et al. Factors contributing to anemia after uncomplicated falciparum malaria. Am J Trop Med. Hyg. 2001; 65:614-622.
39. Looareesuwan S, Ho M, Wattanagoon Y, et al. Dynamic alteration in splenic function during acute falciparum malaria. N Engl J. Med. 1987; 317:675-679.
40. Wyler D J. The spleen in malaria. Ciba Found Symp. 1983; 94:98-116.
41. Dondorp A M, Omodeo-Sale F, Chotivanich K, Taramelli D, White N J. Oxidative stress and rheology in severe malaria. Redox Rep. 2003; 8:292-294.
42. Schellenberg D, Menendez C, Kahigwa E, et al. African children with malaria in an area of intense Plasmodium falciparum transmission: features on admission to the hospital and risk factors for death. Am J Trop Med. Hyg. 1999; 61:431-438.
43. Arditi M, Frinking P J, Zhou X, Rognin N G. A new formalism for the quantification of tissue perfusion by the destruction-replenishment method in contrast ultrasound imaging. IEEE Trans Ultrason Ferroelectr Freq Control. 2006; 53:1118-1129.
44. Gysin J, Fandeur T. (1983). Saimiri sciureus (karyotype 14-7): an alternative experimental model of Plasmodium falciparum infection. Am J Trop Med. Hyg. 32(3):461-7.

45. Gay F, Robert C, Pouvelle B, Peyrol S, Scherf A, Gysin J. (1995). Isolation and characterization of brain microvascular endothelial cells from Saimiri to monkeys. An in vitro model for sequestration of Plasmodium falciparum-infected erythrocytes: J Immunol Methods. 184(1):15-28.
46. Hardeman M R, Goedhart P T, Dobbe J G G, Lettinga K P. (1994). Laser-assisted optical rotational cell analyser (LORCA). 1. A new instrument for measurement of various structural hemorheologic parameters. Clin Hemorheol. 14: 605-618.
47. Hardeman M R, Goedhart P T, Schut N H. (1994). Laser assisted Optical Rotational Cell Analyser. (L.O.R.C.A.) Red blood cell deformability: Elongation index versus cell transit time. Olin Hemorheol. 14(4): 619-630.
48. Rosse W F. The spleen as a filter. N Engl J Med. 1987; 317:704-706.
49. Perrotta S, Gallagher P G, Mohandas N. Hereditary spherocytosis. Lancet. 2008; 372:1411-1426.
50. Mohandas N, Gallagher P G. Red cell membrane: past, present, and future. Blood. 2008; 112:3939-3948.
51. Chen L T, Weiss L. The role of the sinus wall in the passage of erythrocytes through the spleen. Blood. 1973; 41:529-537.
52. Miller L H, Good M F, Milon G. Malaria pathogenesis. Science. 1994; 264:1878-1883.
53. Alano P. Plasmodium falciparum gametocytes: still many secrets of a hidden life. Mol. Microbiol. 2007; 66:291-302.
54. Safeukui I, Correas J M, Brousse V, et al. Retention of Plasmodium falciparum ring-infected erythrocytes in the slow, open microcirculation of the human spleen. Blood. 2008; 112:2520-2528.
55. Antia M, Herricks T, Rathod P K. Microfluidic approaches to malaria pathogenesis. Cell Microbiol. 2008; 10:1968-1974.
56. Smilkstein M, Sriwilaijaroen N, Kelly J X, Wilairat P, Riscoe M. Simple and inexpensive fluorescence-based technique for high-throughput antimalarial drug screening. Antimicrob Agents Chemother. 2004; 48:1803-1806.
57. Bennett T N, Paguio M, Gligorijevic B, et al. Novel, rapid, and inexpensive cell-based quantification of antimalarial drug efficacy. Antimicrob Agents Chemother. 2004; 48:1807-1810.
58. Johnson J D, Dennull R A, Gerena L, Lopez-Sanchez M, Roncal N E, Waters N C. Assessment and continued validation of the malaria SYBR green I-based fluorescence assay for use in malaria drug screening. Antimicrob Agents Chemother. 2007; 51:1926-1933.
59. Bacon D J, Latour C, Lucas C, Colina O, Ringwald P, Picot S. Comparison of a SYBR green I-based assay with a histidine-rich protein II enzyme-linked immunosorbent assay for in vitro antimalarial drug efficacy testing and application to clinical isolates. Antimicrob Agents Chemother. 2007; 51:1172-1178.
60. Rason M A, Randriantsoa T, Andrianantenaina H, Ratsimbasoa A, Menard D. Performance and reliability of the SYBR Green I based assay for the routine monitoring of susceptibility of Plasmodium falciparum clinical isolates. Trans R Soc Trop Med. Hyg. 2008; 102:346-351.
61. Trager W, Jensen J B. Human malaria parasites in continuous culture. Science. 1976; 193:673-675.

1. A method for screening compounds for their ability to increase rigidity of red blood cells (RBC) infected by a protozoan parasite of the genus Plasmodium, said method comprising or consisting of the following steps: a) culturing RBCs infected by said parasite and optionally and separately culturing uninfected RBCs, each culture being carried out both in the presence and in the absence of a compound to be tested for its ability to increase an in particular selectively increase rigidity of infected RBCs (iRBCs) and; b) measuring the deformability of one or several iRBCs cultured in the presence of said compound, and one or several iRBCs cultured in the absence of said compound; and, c) optionally, measuring the deformability of one or several uninfected RBCs cultured in the presence of said compound, and one or several uninfected RBCs cultured in the absence of said compound, wherein a decrease by at least 5%, preferably at least 10% and more preferably at least 15%, of the deformability of iRBCs cultured in the presence of a compound in comparison with the deformability of iRBCs cultured in the absence of the same compound is indicative that said compound is able to increase rigidity of iRBCs.

2-19. (canceled)

20. A method for filtering red blood cells (RBCs) that enables retention, in a filtering unit, of RBCs having an abnormal and in particular a decreased deformability, said method comprising or consisting of the following steps:
a) allowing a sample comprising RBCs to flow through a filtering unit; and
b) retrieving an aliquot of said sample before it flows through the filtering unit (upstream aliquot) and an aliquot of said sample after it has flown through the filtering unit (downstream aliquot); and
c) optionally retrieving the RBCs that have been retained into the filtering unit (retained aliquot); and
d) optionally analyzing the upstream and downstream aliquots and, optionally, the retained aliquot, and in particular determining the density of RBCs or of a RBC sub-population in the upstream and downstream aliquots, and, optionally, in the retained aliquot.

21-46. (canceled)

47. A method for filtering red blood cells (RBCs) that enables retention, in a filtering unit, of RBCs having an abnormal and in particular a decreased deformability, said method comprising or consisting of the following steps:
a) allowing a sample comprising RBCs to flow through a filtering unit; and
b) optionally, retrieving an aliquot of said sample before it flows through the filtering unit (upstream aliquot) and an aliquot of said sample after it has flown through the filtering unit (downstream aliquot); and
c) optionally, retrieving the RBCs that have been retained into the filtering unit (retained aliquot); and
d) optionally, analyzing the upstream and downstream aliquots and, optionally, the retained aliquot, and in particular determining the density of RBCs or of a RBC sub-population in the upstream and downstream aliquots, and, optionally, in the retained aliquot,
wherein said RBCs are for example human RBCs.

48. The method according to claim 47, wherein step d) further comprises calculating (i) the RBC retention rate in the filtering unit, for example using the following formula: (density of RBCs or of a RBC sub-population in the downstream aliquot—density of RBCs or of a RBC sub-population in the upstream aliquot)/density of RBCs or of a RBC sub-population in the upstream aliquot, or (ii) the RBC retention rate in the filtering unit, using the following formula: density of RBCs or of a RBC sub-population in the downstream aliquot/density of RBCs or of a RBC sub-population in the upstream aliquot.

49. The method according to claim 47, wherein step d) comprises determining haemolysis in the upstream and downstream aliquots, and, optionally, in the retained aliquot, for example by quantifying human lactate dehydrogenase (LDH) concentration in the supernatant from the centrifuged upstream and downstream aliquots, and, optionally, from the centrifuged retained aliquot.

50. The method according to claim 47, wherein the sample comprises RBCs that have a decreased deformability and in particular RBCs chosen among:
RBCs infected by a parasite, preferably a protozoan parasite of the genus Plasmodium, and in particular ring-hosting iRBCs or gametocyte-hosting iRBCs, for example mature gametocyte-hosting iRBCs, said parasite being preferably chosen in the group consisting of Plasmodium falciparum, Plasmodium vivax, Plasmodium ovale, Plasmodium malariae, Plasmodium knowlesi, Plasmodium inui, Plasmodium cynomolgi, Plasmodium simiovale, Plasmodium brazilianum, Plasmodium schwetzi and Plasmodium simium, preferably in the group consisting of Plasmodium falciparum, Plasmodium vivax, Plasmodium ovale, Plasmodium knowlesi, and Plasmodium malariae, and more preferably in the group consisting of Plasmodium falciparum and Plasmodium vivax, for example a Plasmodium falciparum of the Palo Alto I strain;
heated RBCs, and
RBCs from a patient who may have inherited or acquired immune dysfunctions as well as inherited or acquired RBC disorders, and preferably from a patient who may have hereditary or acquired spherocytosis, elliptocytosis, sepsis, hemoglobinopathies (alpha or beta thalassemia, sickle cell disease and sickle cell trait), auto immune hemolytic anaemia, other hemolytic anaemias, enzyme deficiencies (Glucose 6 Phosphate Deshydrogenase, Pyruvate Kinase, other red blood cell enzyme).

51. The method according to claim 47, wherein the RBCs which are analyzed in step d) or at least one RBC sub-population which is analyzed in step d) is(are) abnormal RBCs or Plasmodium-infected RBCs (iRBCs), and in particular ring-hosting iRBCs or gametocyte-hosting iRBCs, for example mature gametocyte-hosting iRBCs.

52. The method according to claim 51, wherein step d) comprises or consists in:
determining the percentage of parasitaemia in the upstream and downstream aliquots, and, optionally, in the retained aliquot; and
optionally calculating (i) the RBC retention rate in the filtering unit using the following formula: (percentage of parasitaemia in the downstream aliquot—percentage of parasitaemia in the upstream aliquot)/percentage of parasitaemia in the upstream aliquot or (ii) the RBC retention rate in the filtering unit, using the following formula: percentage of parasitaemia in the downstream aliquot/percentage of parasitaemia in the upstream aliquot.

53. The method according to claim 47, wherein:
RBC of the sample have previously been exposed to a compound, in particular a compound that may be able to modulate and preferably to increase rigidity of RBCs, and for example a compound that may specifically modulate and preferably increase rigidity of iRBCs; and
preferably, wherein RBCs have previously been exposed to said compound in vitro, e.g., by culturing in vitro RBCs in the presence of said compound.

54. The method according to claim 47, wherein the RBCs have been retrieved from a sample of blood, and preferably from a sample of peripheral blood, previously obtained from a patient.

55. The method according to claim 47, wherein the channels of the filtering unit have:
a diameter in the range of 1 to 10 μm, and preferably in the range of 1.85 to 9.4 μm or 1 to 3 μm, for example a diameter of 2 μm; and/or
a thickness of less than 24 μm, and preferably less than 5 μm.

56. The method according to claim 47, wherein the flow through the filtering unit is driven by gravity, flush, aspiration or by centrifugation.

57. The method according to claim 47, wherein the hematocrit of the sample is low, for example less than 5%, and more preferably in the range of 2%-2.5%, said sample being preferably suspended in a suspending medium comprising, consisting of or consisting essentially of PBS or RPMI supplemented with 4% albumin and 5% Plasmion® or with 1% albumax II®.

58. The method according to claim 47, wherein the filtering unit comprises or consists of channel-perforated membrane(s), for example polycarbonate channel-perforated membrane, and wherein optionally step a) is gravity driven and performed under a constant pressure, for example a constant pressure of 80-85 cm of water, and preferably at a temperature of about 34-37° C.

59. The method according to claim 47, wherein:
the filtering unit comprises or consists of one or several layer(s) of beads, and preferably of tin beads, wherein beads present in the filtering unit have a diameter in the range of 2-25 μm or 5-25 μm, wherein channels formed by the inter-bead space within the filtering unit preferably varies between 0.74 and 9.4 μm or 1.85 μm and 9.4 μm, and wherein, optionally, each layer of beads present in the filtering unit is at least 0.5-10 μm thick, the total thickness of beads in the filtering unit being of at least 5 mm, preferably 7 mm; and
optionally, step a) is performed under a constant pressure, for example a constant pressure of 80-85 cm of water, and preferably at a temperature of about 20-25° C.

60. The method according to claim 59, wherein the flow through the filtering unit is a syringe-pressured flow or a flow induced by centrifugation.

61. The method according to claim 47, wherein step d) and in particular density or parasitemia determinations are performed by a liquid-phase fluorescence-based quantification method or after Giemsa staining.

62. The application of the method for filtering RBCs according to claim 47, for isolating and/or detecting RBCs with abnormal and especially reduced deformability, in particular for experimental studies and/or for the diagnosis or detection of the presence of Plasmodium-infected RBCs, for example ring-hosting RBCs and/or gametocytes-hosting RBCs, or for the diagnosis or prognosis of acquired or inherited disorders that affect RBC deformability, for example acquired or hereditary spherocytosis.

63. The application of the method for filtering RBCs according to claim 47, for the in vitro analysis of spleen function in a patient, and in particular in a patient with inherited or acquired immune dysfunctions as well as inherited or acquired RBC disorders.

64. A method for screening compounds for their ability to increase rigidity of red blood cells (RBC) infected by a protozoan parasite of the genus Plasmodium, said method comprising or consisting of the following steps:
a) culturing RBCs infected by said parasite and optionally separately culturing uninfected RBCs, each culture being carried out both in the presence and in the absence of a compound to be tested for its ability to increase and in particular selectively increase rigidity of infected RBCs (iRBCs) and;
b) measuring the deformability of one or several iRBCs cultured in the presence of said compound, and one or several iRBCs cultured in the absence of said compound; and,
c) optionally, measuring the deformability of one or several uninfected RBCs cultured in the presence of said compound, and one or several uninfected RBCs cultured in the absence of said compound,
wherein a decrease by at least 5%, preferably at least 10% and more preferably at least 15%, of the deformability of iRBCs cultured in the presence of a compound in comparison with the deformability of iRBCs cultured in the absence of the same compound is indicative that said compound is able to increase rigidity of iRBCs,
wherein said uninfected and infected RBCs are for example human RBCs, and wherein the screening performed is preferably a low throughput screening or a high throughput screening.

65. The method according to claim 64, wherein said iRBCs are for example ring-hosting RBCs and/or gametocytes-hosting RBCs, in particular mature gametocytes-hosting RBCs.

66. The method according to claim 64, wherein the iRBCs used at step a) where obtained by the following step:
infecting RBCs, preferably RBCs of less than 8 days of age, with iRBCs, in particular with a culture of iRBCs in which the schizont-stage iRBCs have been enriched, said enrichment being performed for example by treatment with a gelatinous solution, for example Plasmion®.

67. The method according to claim 64, wherein step a) and/or the step of claim 20 is (are) preceded by the following step:
synchronization of the parasites of the iRBCs or of the parasites of the schizont-enriched iRBCs culture, for example by one or several sorbitol treatment(s).

68. The method according to claim 64, wherein one screens the deformability of uninfected RBCs cultured in the presence of the compound which is approximately the same or differs by less than 5% from that of the uninfected RBCs cultured in the absence of the compound.

69. The method according to claim 65, wherein one screens the deformability of schizont-iRBCs cultured in the presence of the compound which is approximately the same or differs by less than 5% from that of the uninfected schizont-iRBCs cultured in the absence of the compound.

70. The method according to claim 64, wherein the deformability of RBCs and iRBCs is measured on a populational cellular scale and/or on an individual cellular scale.

71. The method according to claim 64, wherein the deformability of RBCs and iRBCs is measured:
by means of a rheoscope, an ektacytometer, a microfluidic device, a micropipet and/or an optical tweezer, the ektacytometer being for example a commercial automated instrument, preferably a Laser-assisted Optical Rotational Cell Analyzer (LORCA; Mechatronics, Hoorn, Netherlands) or a RHEODYN-SSD (Myrenne, Roetgen, Germany), and more preferably a LORCA; and
preferably over a range of shear stresses from 0.3 to 30 Pascal (Pa), including 1.7 Pa, and 30 Pa.

72. The method according to claim 64, wherein the parasitemia level of iRBCs is of at least 20% or 30% of the cell population, preferably at least 50% and more preferably at least 70% of the cell population.

73. The method according to claim 64, wherein the measure of deformability is carried out by reference to the elongation index (EI), and wherein preferably an EI below 0.43, more preferably below 0.42, for iRBCs cultured in the presence of the compound, when extrapolated at 100% parasitemia, at 30 Pa, is indicative that said compound is able to increase rigidity of iRBC.

74. The method according to claim 64, wherein said parasite is chosen in the group consisting of Plasmodium falciparum, Plasmodium vivax, Plasmodium ovale, Plasmodium malariae, Plasmodium knowlesi, Plasmodium inui, Plasmodium cynomolgi, Plasmodium simiovale, Plasmodium brazilianum, Plasmodium schwetzi and Plasmodium simium, preferably in the group consisting of Plasmodium falciparum, Plasmodium vivax, Plasmodium ovale, Plasmodium knowlesi, and Plasmodium malariae, and more preferably in the group consisting of Plasmodium falciparum and Plasmodium vivax, for example wherein said parasite is the Palo Alto I strain of Plasmodium falciparum.

75. The method according to claim 64, further comprising a step of validating a screened compound with the isolated perfused human spleen model and/or an isolated perfused pig spleen model.

76. The method according to claim 64, wherein the screened compounds are capable of interacting with the iRBCs membrane its cortical cytoskeleton included and/or entering into the iRBCs, in particular crossing the iRBCs lipid bilayer, or interact with the modified bilayer itself.

77. The application of the method according to claim 64, for the selection of compounds which selectively interact with red blood cells (RBCs) infected by a protozoan parasite of the genus Plasmodium (iRBCs) or selectively interact with iRBCs at the ring stage and/or gametocytes-hosting RBCs, in particular mature gametocytes-hosting RBCs, and are suitable to increase their rigidity.

78. The application of the method according to claim 47, in a method for screening compounds for their ability to modulate deformability and in particular to induce or increase rigidity of red blood cells (RBCs) and especially of RBCs infected by a protozoan parasite of the genus Plasmodium, for example in a method according to claim 64 or in a method of screening comprising or consisting of the following steps:
applying the method for filtering RBCs according to claim 47, to two aliquots of a sample comprising RBCs, wherein only one aliquot has previously been exposed to a compound to be tested for its ability to modulate and preferably to increase rigidity of RBCs; and
comparing the RBC retention rate or retention ratio determined for both sample aliquots, wherein a variation of at least a 5%, preferably at least 10% and more preferably at least 15% between both RBC retention rates or both retention ratio is indicative that said compound is able to modulate rigidity of RBCs and wherein an increase by at least 5%, preferably at least 10% and more preferably at least 15%, in the RBC retention rate (in absolute value) or in the retention ratio obtained for the sample aliquot that has been exposed to the compound in comparison with the RBC retention rate (in absolute value) or the retention ratio obtained for the sample aliquot that has not been exposed to the compound is indicative that said compound is able to increase rigidity of RBCs.

79. The method of screening according to claim 64, wherein the measure of deformability of RBCs or iRBCs is carried out using the method for filtering RBCs according to claim 47, preferably by reference to the RBC or iRBC retention rate or retention ratio.
